SEQUENCE LISTING (26 sequences)

SEQ ID NO: 1 - DNA, 1194 bp, Alternaria destruens
  source 1..1194: mol_type = other DNA
tacatcctcttctaccgcgacacccgaaccaaccctcacgccgagcaaaccaccaagaagaaggcctggtggcagttctggaagtctggctcagctaccgctgccactcccatccaggatgccggtgccgtccccgacgactgtaagttttatcatcctgctcactcgattgcatgcacctgcatcacatagcactgctgtttgcggcagcgctcaacgtacctcgccaattcatcctttgttgagctttacctcgacatttggtggctggcatggtccgcgctcaagctgctccctgctagcgacgcgatagcggcagaaatggtggagccaatcatgcaatccggctccaccaaactacccgcttctgcagcatccgaaatgagcaacacgatcaagaggaattttgctaacatggaattgcagacctcaacactgagctccgaactggtctcacctcgtccgacgttgagcagcgtcgcaagcgctatggtttcaacgaaatctcttctgagaagaccaaccttctcaagcagttcatcggttacttcactggtcccattctctacggtaagcatccctgcacaaacttgtttagcgccaaactaacgcatcatagtcatggagctcgctgctcttctcgccgctggtcttcaggattgggtcgatttcggtgtcatctgcggtatcctgttgctcaacgccatcgtcggttggtaccaggagaaacaggctgctgatgtcgtcgcttcgctcaagggtgatatcgccatgaaggccaccgtcgttcgtgacaaccagcaacagaccattctcgctcgtgagcttgttcccggtgacatcgtcgttattgaggagggtcaatccgtccccggtgacgcccgtcttatctgcggctacgaccaccctgaggacttcgacttgtacatgaagctcaaggctgaggacaagttccacgacgctgaccccgaggacgagaaggatgacgacgtcgatgaggagaagttcgacgaggagaaccccatcactcagggccaccctctcgttgcttgcgatcaatcgtccatcaccggagagtctctcgctgtcgacaagtacatgggagaagtcgcctactacaccactggttgcaagcgcggcaaggcctacggtatcgtcatcaccactgctaagcactctttcgtcggtcgcact

SEQ ID NO: 2 - DNA, 775 bp, Alternaria destruens
  source 1..775: mol_type = other DNA
cgtaagtgccctccccatcctctgccatgccgcgcggctgcctggtagccctgggggcctgcgcaatcacgaacatgcagctgacgacgtcgtgttgtaggacaaggatggcgatggtcagtactctccctccaaattcccttccacacacacactctctctccctctctgccttcaaagcagtgccgcatctccagcctacgcaatcggcagaggggcccgggcgaggcttgctggctaggggtccaaaccaccgcccacagctacaacaccacgacatccaccctactccatagcaagcacaactgacgacgatgcgccacaggtcaaatcaccaccaaggagctaggtaccgtcatgcgctcgctcggccaaaatcccagcgagtctgagctccaggacatgatcaacgaggtcgatgccgacaacaacggcaccattgacttcccaggtgcgccccttcataccagtccaaagtaccacagctaacttttccagaattccttaccatgatggcccgcaagatgaaggacaccgactccgaggaggagatccgggaagccttcaaggtcttcgaccgcgataacaacggtttcatctccgccgccgaactgcgtcacgtcatgacttctattggcgagaaattgaccgatgacgaggtcgacgagatgatccgggaggctgaccaggacggtgacggccgcatcgactgtaggttacagctgcctatatcacgagtgcgatgctaacacacatcagacaacgagttcgtcca

SEQ ID NO: 3 - DNA, 1563 bp, Fusarium commune
  source 1..1563: mol_type = other DNA
ttttcctcacaaaggagcaaatcatgaactgtatgctctgggtgcctaactgggacggtgtcattcctcaacccgctatctataagcctcgtcctcggtggactggtaagcagcttatcagcatggttatccctaaggaggttagcctgttcaacggtacggattctggtgaaaacgcccctcttaaggacgagggtcttctgatccaagctggccaactgatgtatggtcttttgactaagaagaacattggtgctgctgcgggcggtattgtgcatatcagctacaacgaacttggccccgaaggtgcgatggctttcttgaacggtgtccagcaggttgtcacctactggcttctcaacaatggccatagcattggtattggtgatacaattcccgatgcggcgaccattgctaaagttcaggtacatattgatgaggaaaaggctgaagttgctcgcttgacagcaatggccacagcgaatgagcttgaggccctacctggtatgaacgttcgtgcaaccttcgaaaacaaggtctccatggctctgaaccaggcccgtgataaggctggtaccacaacacagaagagtttgaaggattcaaacaacgctgtcaccatggcttcctcaggttccaagggttcatctatcaatatttctcaaatgactgcgcttgtcggtcagcaaattgtcgaaggcaagcgtattccttttggtttcaagtatcgcacattacctcacttcaccaaggacgattactcacctgaggcccgtggcttcgtcgagaactcttacctccgtggtctcactcccagcgagtttttcttccacgccatggctggtcgagaaggtctcattgatactgcagtcaagactgccgaaacaggttatatccagcgacgattggtcaaggctctggaagatctttctgcccgttacgatggaactgtccgaaactctctgggagacattgttcagttcctctatggtgaagacggtctcgatgccatgattattgagaaacagaagttgggtatcctcaatatgtcaaactcggcatttgaaaagaagtatcgtctggatcttgccaaccccccggactggtttaagcacgactacgaattcggtaatgaattgactggtgacaaggaatctatggagtatctcgatcaagaatgggaaaagttgttggctgatcgcagacaaatccgacagatcaacaaggccaagggtaacgaggaaatgatgcaactgcccctcaacatcactcgcatcatcgagtctgctaagcgagtctttaatgtcaaggctaatgaccgaagcaacttgcgaccttcggaagttattccagctgtgcaaaacttgttggatagcatgaagattgttcgtggtactgatgaaatctcgattgaagctgacgcaaatgcatccattctcttcaaggccttgcttcgctctcgcctggccttcaaggaggtggtcaaggagcaccggttgaacaaattagctttcgaccatattctgggtgaactccagaatagatgggatcgcgcatttgtca

SEQ ID NO: 4 - DNA, 1889 bp, Fusarium commune
  source 1..1889: mol_type = other DNA
ttgacagatatacctttgcttcgactctttcacatttgcgtcgaaccaatactcctattggacgagatggtaaattggccaagcctcgacagcttcacaatactcactggggtttggtgtgtcccgccgaaacgcctgagggtcaagcttgtggtctggtcaaaaacttgtctctgatgtgttatgtcagtgtcggctctccagccgagcctctcattgaattcatgatcaacagaggtatggaagttgttgaggagtacgagccgacaagatatccccacgctacaaagattttcgtcaacggtagctgggttggtgttcacgccgaccccaagcatctcgtgaatcaggttttggacacaagacgaaagtcgtacgttcagttcgaagtatcacttgttcgtgatattcgagaccgtgaattcaagattttctcagacgctggtcgtgtcatgagacccgtctttacagtccatcaggaggatgactatgagaacaacatcaccaagggacaactagtgttgacaaaggaacatgtcaataggctagcccaagagcaggcagagccacctgccaaccccgcggacaagtttggatgggatggcttgattcgcgaaggagctgtcgagtatctcgacgctgaggaagaagagacagccatgatttgcatgacgccagaggatctcgaactttaccgtgagcaaaagaatgatgaagctacactcacggaagaagagaaacgggccaaggcagaggcagagaagagggaacaagaggaggaccgcaacaagcgattgaagacaaaggtcaaccccacaactcacatgtacacacattgtgagattcaccccagtatgattctcggtatctgtgccagtatcattcctttccccgatcacaaccaggtatgtagtccctttgatcacaacaacctcaacnnnnnnnnnnnnnnnnnnnnnnnnnnnnnnnnnnnnnnnnnnnnnnnnnnnnncaatattctctactaccctcaaaagcctctcgccactacccgatccatggagttcctcaagttccgtgaattgccagctggtcaaaatgccattgtcgcaattgcttgctactcaggttataaccaggaagattccgtcattatgaaccagagtagtattgatcgaggtctgttccgaagtctgttcttccgatcgtactcagaccaggagaagaaggtcggcctcaactacactgagatctttgagaagcctttccagcagacaacactccgaatgaagcatggaacatacgacaagcttgacgaggatggtatcgtggctcctggtgtccgtgtgtctggtgaagatatcattatcggcaagactgcacccatcgaccaagaaaaccaggaccttggcacaaggactcaatcgcaccagcgtcgtgatatctcgacaccactgcgaagtaccgagaacggtatcgttgatcaggtcattctgacagtcaacgccgataacgtcaagtacgtcaaggttcgagtacgaacaaccaagattcctcaaatcggtgacaagtttgcttctcgtcacggtcaaaagggtacaatcggtgttacatatcgacaggaggatatgcctttcagccgagaaggtctcacccccgatatcattatcaaccctcacgccattccatcgcgaatgacaattgcccatttgattgagtgtctccttagcaaggtttcaacgctggaaggtatggagggtgacgccacaccgttcactgatgtcacagtcgattcagtctcagaacttctgaggaagcacggttaccaatctcgaggtttcgaggtcatgtacaatggtcacactggacgaaagctccgtgcccaggtgttcttcggacctacctactac

SEQ ID NO: 5 - DNA, 678 bp, Fusarium commune
  source 1..678: mol_type = other DNA
gactcaccttaacgtcgtcgtcatcggccacgtcgactctggcaagtcgaccactgtgagtactccccttggacgatgagcttatctgccatcgttaatcccgaccaagacctggcggggtatttctcaaaggcaatatgctgatatcgtttcacagaccggtcacttgatctaccagtgcggtggtatcgacaagcgaaccatcgagaagttcgagaaggttagtcactttcccttcgatcgcgcgtcctctgcccatcgatttcccctacgactcgaaacctgcccgctaccccgctcgagaccaaaaattttgcgatatgaccgtaattttttttggtggggcatttaccccgccactcgagcgacgggcgcgtttgccctcctcccatttccacaacctcaatgagcgcatcgtcacgtgtcacgcagtcactaaccattcaataataggaagccgctgagctcggtaagggttccttcaagtacgcctgggttcttgacaagctcaaggccgagcgtgagcgtggtatcaccatcgatattgctctctggaagttcgagactcctcgctactatgtcaccgtcattggtatgttgtcgctcatgcttcattctacttctcttcgtactgacatatcactcagacgctcccggtcaccgtgatttcatcaagaa

SEQ ID NO: 6 - DNA, 1470 bp, Fusarium oxysporum
  source 1..1470: mol_type = other DNA
ttttcctcacaaaggagcaaatcatgaactgtatgctctgggtgcccaactgggacggtgtcattcctcaacccgctatctataagcctcgtcctcggtggactggtaagcagcttatcagcatggttatccctaaggaggttagcctgttcaacggtacggattctggtgaaaacgcccctcttaaggacgagggtcttctgatccaagccggccaactgatgtatggtcttttaactaagaagaacattggtgctgctgcgggtggtattgtgcatatcagctacaacgaacttggccccgaaggtgcgatggctttcttaaacggtgtccagcaggttgtcacctactggcttctcaacaatggtcatagcattggtattggtgatacaattcccgatgcggcgaccattgctaaagttcaggtacatattgatgaggaaaaggctgaagttgcccgcttgacagcaatggccacagcgaatgagcttgaggccctacctggtatgaacgttcgtgcaaccttcgaaaacaaagtctccatggctctgaaccaggcccgtgataaggctggtaccacaacacagaagagtttgaaggattcaaacaacgctgtcaccatggcttcctcaggttccaagggttcatctatcaatatttctcaaatgactgcgcttgtcggtcagcaaattgtcgaaggcaagcgtattccttttggtttcaagtatcgcacattacctcacttcaccaaggacgattactcacctgaggcccgtggcttcgtcgagaactcttacctccgtggtctcactcctagcgaatttttcttccacgccatggctggtcgagaaggtctcattgatactgcagtcaagactgccgaaacaggttatatccagcgacgattggttaaggctctggaagatctttctgcccgttacgatggaactgtccgaaactctctgggagacattgttcagttcctctatggtgaagacggtcttgatgccatgattattgagaaacagaagttgggtatcctcaatatgtcaaactcggcatttgaaaagaagtatcgtctggatcttgccaaccccccggactggtttaagcacgactacgaattcggtaacgaattgactggtgacaaggaatctatggagtatctcgatcaagaatgggaaaagttgttggctgatcgcagacaagtccgacagatcaacaaggccaagggtaacgaggaaatgatgcaactgcccctcaacatcactcgcatcatcgagtctgctaagcgagtctttaatgtcaaggctaatgaccgaagcaacttgcgaccgtcggaagttattccagctgtgcaaaacttgttggatagcatgaagattgttcgtggtactgatgaaatctcgattgaagctgacgcaaatgcatcatttctcttcaaggccttgcttcgctctcgcctggcct

SEQ ID NO: 7 - DNA, 1850 bp, Fusarium oxysporum
  source 1..1850: mol_type = other DNA
tgcttctactctttcacatttgcgtcgaaccaatactcccatcggacgagatggtaaattggccaagcctcgacagcttcacaacactcactggggtttggtgtgtcctgccgaaacacctgagggtcaagcttgtggtctggtcaaaaacttgtctctaatgtgttacgtcagtgtcggctctccagccgatcctctgattgaattcatgatcaacagaggcatggaagtcgttgaggagtacgagccgacaagatacccccacgctacaaagattttcgtcaacggtagctgggttggtgttcatgccgaccccaagcatctcgtgaatcaggtcttggacacaagacgaaagtcttacgtgcagttcgaagtatcacttgttcgtgatatccgagaccgtgaattcaagattttttcagacgctggccgtgtcatgagacccgtctttacagttcatcaggaggatgactatgagaacaacatcaccaagggacaactagtgttgacaaaggaccatgtcaataggctagcccaagaacaggcagagcctcctgccaacccagcggacaagtttggatgggatggcttgatccgcgaaggagctgtcgagtatctcgatgctgaggaagaagagacagccatgatttgcatgacgccagaggatctcgaactttaccgtgagcaaaagaatgatgaagctacactcacagaagaagagaaacgggccaagcaagaggcagagaagagagaacaagaggaggaccgcaacaagcgattgaagacaaaggtgaaccccacaactcacatgtacacacattgtgagattcaccccagtatgattctcggtatctgtgccagtatcattcctttccccgatcacaaccaggtatgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtagtattgatcgaggtttgttccgaagtctgttcttccgatcgtactcagatcaggagaagaaggttggtctcaactacactgagatctttgagaaacctttccagcagacaacgcttcgaatgaagcatggaacatacgacaagcttgatgaagatggtatcgtggctcctggtgtccgtgtgtcaggtgaagatatcattatcggcaagactgcacccatcgaccaagaaaaccaggaccttggcacaagaactcaatcgcaccaacgtcgtgatatctcgacaccactgcgaagtactgagaacggtatcgttgatcaagtcattctgacagtcaacgccgataacgtcaagtacgtcaaggtccgagtacgaacaaccaagattcctcaaattggtgacaagtttgcttctcgtcacggtcaaaagggtacaatcggtgttacatatcgacaggaggatatgcctttcagccgagaaggtcttactcccgatatcattatcaaccctcacgccattccatcgcgaatgacaattgcccatttgattgagtgtcttcttagcaaggtttcaacgctggaaggtatggagggtgacgccacaccgttcactgatgtcacagtcgattcagtctcagaacttctgaggaagcacggttaccaatctcgaggtttcgaggtcatgtacaacggtcacactggacgaaagctccgtgcccagg

SEQ ID NO: 8 - DNA, 687 bp, Fusarium oxysporum
  source 1..687: mol_type = other DNA
agacaagactcaccttaacgtcgtcgtcatcggccacgtcgactctggcaagtcgaccactgtgagtactctccttgacaatgagcttatctgccatcgtcaatcccgaccaagacctggcggggtatttctcaaagtcaacatactgacatcgtttcacagaccggtcacttgatctaccagtgcggtggtatcgacaagcgaaccatcgagaagttcgagaaggttagttactttcccttcgatcgcgcgtcttttgcccatcgatttcccctacgactcgaaacgtgcccgctacccctctcgagaccaaaaattttgcaatatgaccgtaatttttttggtggggcatttaccccgcccctcgggtgccgggcgcgtttgccctcttaccattctcacaacctcaatgagcgcatcgtcacgtgtcaagcagtcactaaccattcaacaataggaagccgctgagctcggtaagggttccttcaagtacgcctgggttcttgacaagctcaaggccgagcgtgagcgtggtatcaccatcgatattgctctctggaagttcgagactcctcgctactatgtcaccgtcattggtatgttgtcgctcatgcttcattctacttctcttcgtactaacacatcactcagacgctcccggtcaccgtgatttcatcaagaacatga

SEQ ID NO: 9 - DNA, 20 bp, synthetic construct
  source 1..20: mol_type = other DNA
atgggtaaggargacaagac

SEQ ID NO: 10 - DNA, 19 bp, synthetic construct
  source 1..19: mol_type = other DNA
ggargtaccagtsatcatg

SEQ ID NO: 11 - DNA, 22 bp, synthetic construct
  source 1..22: mol_type = other DNA
cayaargartcyatgatgggwc

SEQ ID NO: 12 - DNA, 22 bp, synthetic construct
  source 1..22: mol_type = other DNA
caatgagaccttctcgaccagc

SEQ ID NO: 13 - DNA, 21 bp, synthetic construct
  source 1..21: mol_type = other DNA
cracacagaagagtttgaagg

SEQ ID NO: 14 - DNA, 22 bp, synthetic construct
  source 1..22: mol_type = other DNA
tcargcccatgcgagagttgtc

SEQ ID NO: 15 - DNA, 19 bp, synthetic construct
  source 1..19: mol_type = other DNA
ggggwgaycagaagaaggc

SEQ ID NO: 16 - DNA, 20 bp, synthetic construct
  source 1..20: mol_type = other DNA
cccatrgcttgyttrcccat

SEQ ID NO: 17 - DNA, 20 bp, synthetic construct
  source 1..20: mol_type = other DNA
atgggyaarcaagcyatggg

SEQ ID NO: 18 - DNA, 21 bp, synthetic construct
  source 1..21: mol_type = other DNA
gcrtggatcttrtcrtcsacc

SEQ ID NO: 19 - DNA, 22 bp, synthetic construct
  source 1..22: mol_type = other DNA
atcgtctccatgaccgagttcg

SEQ ID NO: 20 - DNA, 22 bp, synthetic construct
  source 1..22: mol_type = other DNA
tccgatggagttcatgatagcc

SEQ ID NO: 21 - DNA, 21 bp, synthetic construct
  source 1..21: mol_type = other DNA
agcaagtctccgagttcaagg

SEQ ID NO: 22 - DNA, 22 bp, synthetic construct
  source 1..22: mol_type = other DNA
cttctgcatcatcayctggacg

SEQ ID NO: 23 - protein, 287 aa, Alternaria destruens
YILFYRDTRTNPHAEQTTKKKAWWQFWKSGSATAATPIQDAGAVPDDYLNTELRTGLTSSDVEQRRKRYGFNEISSEKTNLLKQFIGYFTGPILYVMELAALLAAGLQDWVDFGVICGILLLNAIVGWYQEKQAADVVASLKGDIAMKATVVRDNQQQTILARELVPGDIVVIEEGQSVPGDARLICGYDHPEDFDLYMKLKAEDKFHDADPEDEKDDDVDEEKFDEENPITQGHPLVACDQSSITGESLAVDKYMGEVAYYTTGCKRGKAYGIVITTAKHSFVGRT

SEQ ID NO: 24 - DNA, 1194 bp, Alternaria destruens
  source 1..1194: mol_type = other DNA
tacatcctcttctaccgcgacacccgaaccaaccctcacgccgagcaaaccaccaagaagaaggcctggtggcagttctggaagtctggctcagctaccgctgccactcccatccaggatgccggtgccgtccccgacgactgtaagttttaccatcctgctcactcgattgcatgcacctgcatcacatagcactgctgtttgcggcagcgctcaacgtacctcgccaattcatcctttgttgagctttacctcgacatttggtggctggcatggtccgcgctcaagctgctccctgctagcgacgcgatagcggcagaaatggtggagccaatcatgcaatccggctccaccaaactacccgcttctgcagcatccgaaatgagcaacacgatcaagaggcattttgctaacatggaattgcagacctcaacactgagctccgaactggtctcacctcgtccgacgttgagcagcgtcgcaagcgctatggtttcaacgaaatctcttctgagaagaccaaccttctcaagcagttcatcggttacttcactggtcccattctctacggtaagcatccctgcacaaacttgtttagcgccaaactaacgcatcatagtcatggagctcgctgctcttctcgccgctggtcttcaggattgggtcgatttcggtgtcatctgcggtatcctgttgctcaacgccatcgtcggttggtaccaggagaaacaggctgctgatgtcgtcgcttcgctcaagggtgatatcgccatgaaggccaccgtcgttcgtgacaaccagcaacagaccattctcgctcgtgagcttgttcccggtgacatcgtcgttattgaggagggtcaatccgtccccggtgacgcccgtcttatctgcggctacgaccaccctgaggacttcgacttgtacatgaagctcaaggctgaggacaagttccacgacgctgaccccgaggacgagaaggatgacgacgtcgatgaggagaagttcgacgaggagaaccccatcactcagggccaccctctcgttgcttgcgatcaatcgtccatcaccggagagtctctcgctgtcgacaagtacatgggagaagtcgcctactacaccactggttgcaagcgcggcaaggcctacggtatcgtcatcaccactgctaagcactctttcgtcggtcgcact

SEQ ID NO: 25 - DNA, 767 bp, Alternaria destruens
  source 1..767: mol_type = other DNA
cgtaagtgccctccccatcctctgccatgccgcgcggctgcctggtagccctgggggcctgcgcaatcacgaacatgcagctgacgacgtcgtgttgtaggacaaggatggcgatggtcagtactctccctccaaattcccttccacacacacactctctctccctctctgccttcaaagcagtgccgcatctccagcctacgcaatcggcagaggggcccgggcgaggcttgctggctaggggtccaaaccaccgcccacagctacaacaccacgacatccaccctactccatagcaagcacaactgacgacgatgcgccacaggtcaaatcaccaccaaggagctaggtaccgtcatgcgctcgctcggccaaaatcccagcgagtctgagctccaggacatgatcaacgaggtcgatgccgacaacaacggcaccattgacttcccaggtgcgccccttcataccagtccaaagtaccacagctaacttttccagaattccttaccatgatggcccgcaagatgaaggacaccgactccgaggaggagatccgggaagccttcaaggtcttcgaccgcgataacaacggtttcatctccgccgccgaactgcgtcacgtcatgacttctattggcgagaaattgaccgatgacgaggtcgacgagatgatccgggaggctgaccaggacggtgacggccgcatcgactgtaggttacagctgcctatatcacaagtgcgatgctaatacacaccagacaacgag

SEQ ID NO: 26 - protein, 121 aa, Alternaria destruens
DKDGDGQITTKELGTVMRSLGQNPSESELQDMINEVDADNNGTIDFPEFLTMMARKMKDTDSEEEIREAFKVFDRDNNGFISAAELRHVMTSIGEKLTDDEVDEMIREADQDGDGRIDYNE

(19) United States
(12) Patent Application Publication
(10) Pub. No.: US 2023/0292766 A1
(43) Pub. Date: Sep. 21, 2023

(54) Biocontrol of Fusarium by Endophytic Fungi

(71) Applicant: Board of Trustees of Michigan State University, East Lansing, MI (US)

(72) Inventors: Frances Trail, Mason, MI (US); Zachary A. Noel, Auburn, AL (US); Ludmilla V. Roze, Okemos, MI (US)

(21) Appl. No.: 18/184,496

(22) Filed: Mar. 15, 2023

Related U.S. Application Data
(60) Provisional application No. 63/319,874, filed on Mar. 15, 2022.

(51) Int. Cl. A01N 63/30; C12N 1/14; A01P 3/00

(52) U.S. Cl. CPC A01N 63/30; C12N 1/145; A01P 3/00; C12R 2001/77

(57) ABSTRACT

Described herein are methods and compositions that reduce the spread and/or mycotoxin production of Fusarium graminearum. The methods and compositions involve use of one or more of the following endophytes: Alternaria destruens, Fusarium commune, Fusarium oxysporum, or a combination thereof.

### CROSS-REFERENCE TO RELATED APPLICATION

This application claims the priority of U.S. provisional application Ser. No. 63/319,874, filed Mar. 15, 2022, the disclosure of which is incorporated herein by reference in its entirety.

### STATEMENT OF GOVERNMENT SUPPORT

This invention was made with government support under MICL08541 and 59-0206-6-004 awarded by the United States Department of Agriculture. The government has certain rights in the invention.

### INCORPORATION BY REFERENCE OF SEQUENCE LISTING

A Sequence Listing is provided herewith as an xml file, “2317644.xml” created on Mar. 14, 2023, and having a size of 36,234 bytes. The content of the xml file is incorporated by reference herein in its entirety.

### BACKGROUND

Fusarium head blight (FHB) is one of the most devastating diseases of cereals worldwide. The disease is primarily caused by the fungus Fusarium graminearum. The pathogen overwinters on crop residue and perithecia released ascospores which infect wheat heads during anthesis. Infected spikelets bleach and grain is rendered small, shriveled, and discolored. F. graminearum infection can result in grain contamination with the mycotoxins deoxynivalenol (DON) and its derivatives 3-acetyldeoxynivalenol (3ADON) and 15-acetyldeoxyvalenol (15A-DON), the estrogen-mimic zearalenone, aurofusarin (and monomer rubrofusarin), and several others. Importantly, DON also acts as a virulence factor, thus reduction of DON production may also reduce disease. DON and derivatives affect the digestive system and organ function of humans and livestock, resulting in acute emetic effects, as well as more serious health consequences with chronic exposure. Alternatively, F. graminearum can cause root rot, plant stunting, and damping-off. Therefore, the management of this disease would benefit food security and human health.

### SUMMARY

Described herein are methods and compositions for inhibiting the spread of Fusarium spp. as well as mycotoxin production by Fusarium graminearum. The methods and compositions involve use of endophytes such as Fusarium commune, Fusarium oxysporum, and Alternaria destruens. The endophytes can include any of the Fusarium commune, Fusarium oxysporum, or Alternaria destruens conidia, spores, or propagules, or combinations thereof. The endophytes can be administered to plant parts such as roots, buds, flowers, leaves, fruits, seeds, grain heads, or a combination thereof. The endophytes can be administered to whole plants or incorporated into the soil.

With the increase in rainstorms and flooding brought on by climate change, the methods and compositions described herein can reduce mycotoxin contamination without the need for fungicides or pesticides.

### DESCRIPTIONS OF THE FIGURES

FIGS. 1A-1B illustrate the identification of wheat endophytes using phylogenetics. FIG. 1A shows a diagram illustrating the Bayesian phylogeny of Alternaria ATPase and calmodulin genes from Lawrence et al. (Lawrence et al. 2013) with the Alternaria endophyte #37 identified as Alternaria destruens described herein (large arrow). FIG. 1B shows a diagram of the Bayesian phylogeny of RPB1 and RPB2 genes from the Fusarium gibberella clade obtained from O'Donnell et al. (O'Donnell et al. 2013) with Fusarium endophyte #40 (Fusarium commune) and Fusarium endophyte #70 (Fusarium oxysporum) described herein. Bifurcations with posterior probability less than 0.7 do not show the support values.

FIGS. 2A-2B illustrate that the endophytes described herein preemptively restrict the growth of F. graminearum in vitro by occupying space and restricting F. graminearum spread over time. FIG. 2A graphically illustrates the mean surface area of six F. graminearum isolates after physical contact with the endophytes described herein (n=9). As shown, the endophytes exhibit preemptive colonization that restricts the growth of F. graminearum isolates. FIG. 2B shows images illustrating colonization restriction of F. graminearum PH-1 over time by the three endophyte isolates described in the Examples.

FIG. 3 graphically illustrates an increase in dry weight of wheat plants that were grown with F. graminearum (Fg) and a combination of the beneficial fungus Alternaria tenuissima (#37) and Fusarium oxysporum (#70) compared to F. graminearum alone or with F. commune (#40) and a single beneficial fungus (#37 or #70).

### DETAILED DESCRIPTION

Methods and compositions are described herein for inhibiting the spread of Fusarium spp. as well as mycotoxin production by Fusarium spp. Experiments described herein illustrate that endophytes Fusarium commune, Fusarium oxysporum, and Alternaria destruens can inhibit the spread and mycotoxin production by Fusarium graminearum.

Endophytes are microorganisms that live inside plants asymptomatically and can provide protection to the plant against a variety of biotic and abiotic stresses. The endophytes, parts, or propagules can be applied to agricultural crops to reduce the spread of Fusarium spp. as well as mycotoxin production by Fusarium spp. As illustrated herein, endophytes Fusarium commune, Fusarium oxysporum, and Alternaria destruens are effective at inhibiting the spread and mycotoxin production by Fusarium graminearum. Hence, the methods and compositions are generally useful against Fusarium graminearum.

As illustrated in the experimental work described herein, fungal spores from endophytes were produced in culture and harvested. Wheat heads at the beginning of anthesis were pretreated with endophytes by placing inoculum on the rachis between florets. Six days later the heads were challenged with inoculation of spores of F. graminearum, by applying spores inside the floret on the developing seed. After seeds had matured and dried, they were harvested, and seed weight was determined. The levels of the mycotoxins deoxynivalenol and 15-A-deoxynivalenol were measured on the seeds by gas chromatography-mass spectrometry. Three endophytes were shown to have the highest protective activity: Alternaria destruens (#37), Fusarium oxysporum (#70), and Fusarium commune (#40). All three endophytes significantly reduced DON and 15A-DON levels in wheat seeds compared to seed infected with only F. graminearum resulted in near doubling of seed weight when compared to florets inoculated with just F. graminearum.

The endophytes can be applied to agricultural soils, crops, buds, flowers, seeds, fruits, roots, stalks, and leaves. For example, whole plants and crops can be dusted with the endophytes, or the endophytes can be incorporated or applied to soils before planting. The endophytes can be applied, for example, aerially during early grain head emergence to protect from infection and establishment of F. graminearum. It can be useful to apply or implant the endophytes more directly onto or into specific plant tissues such as the flowers, buds, seeds, fruits, roots, or combinations thereof. For example, in some cases, the endophytes can be implanted into vascular tissues, reproductive tissues, root tissues, photosynthetic tissues, and combinations thereof.

Also whole, shredded, or ground endophytes can be used, in some cases endophyte parts (e.g., propagules) can be used. For example, in some cases, the endophyte spores or conidia are more conveniently used in the compositions described herein. The conidia are the asexual, non-motile spores of fungi.

The endophyte spores, conidia, or propagules can be collected by any available method. For example, endophytes can be cultured on media such as MEA (malt extract agar) for a time and under conditions sufficient for conidia/spore formation (e.g., 2-21 days). For example, the spores/conidia can be collected by brushing, rubbing, or washing endophytes growing on the surface of a medium. An implement can be used to rub or brush off the conidia. The implement and any collected spores/conidia can be washed with an aqueous solution. The solution can contain a mild detergent. For example, the conidia/spores can be washed from cultured endophytes, implements, collection vessels, and the like using about 0.01% to 5% mild detergent in water. The collected solution of conidia/spores can be centrifuged at low speeds to separate the spores from debris and/or at higher speeds to sediment them. Conidia, spores, or propagules can be incorporated into liquid or powder compositions that can be applied to agricultural crops.

Plants, seeds, and plant products can be treated with the endophytes described herein. For example, the endophytes described herein can be applied to agricultural plants, seeds, roots, and plant products as well as to soils or any crops for inhibiting mycotoxin production. In addition, plants grown in nature or for decorative purposes can be treated with the compounds and/or compositions described herein. The plants, seeds, and plant products so treated can be for human or animal consumption.

Plants, or seeds from such plants, for example, can be grain-producing, nut-producing, vegetable-producing, fruit-producing, starch-producing, fiber-producing, fodder-producing, or a combination thereof. The plant products can include grains, nuts, vegetables, fruits, starch, fibers, flour, fodder, leaves, stock, seeds, oil, or a combination thereof. For example, the plant products can be almonds, barley, betel nuts, Brazil nuts, cashews, chestnuts, cocoanut, coffee, corn, flour, hazelnuts, macadamia nuts, oats, pecans, peanuts, pine nuts, pistachios, rice, rye, sesame seeds, soybean, spices, walnuts, wheat, or combinations thereof.

Plants can also include vegetables, such as tomatoes, peppers, cabbage, broccoli, asparagus, squash, lettuce, spinach, cauliflower, melon, watermelon, cucumbers, carrots, onions, cucurbits and potatoes, tobacco, pome and stone fruits and berries, such as walnuts, kiwi, banana, avocado, olives, passion fruit, almonds, pineapples, apples, pears, raspberry, cherry, plums, peaches, and cherries, table and wine grapes, citrus fruit, such as oranges, lemons, grapefruits and limes, corn, cotton, soybean, oil seed rape, wheat, barley, rye, triticale, oats, maize, sorghum, sunflower, peanuts, rice, sugar beet, fodder beet, coffee, beans, peas, yucca, sugar cane, clover, turf and ornamentals such as roses.

Additional types of plants, seeds, and plant products can be or can be from flax, cotton, cereals (wheat, barley, rye, oats, millet, triticale, maize (including field corn, popcorn and sweet corn), rice, sorghum and related crops); beet (sugar beet and fodder beet); sugar beet, sugar cane, leguminous plants (beans, lentils, peas, soybeans); oil plants (rape, mustard, sunflowers), Brassica oilseeds such as Brassica napus (e.g. canola), Brassica rapa, B. juncea (e.g. mustard) and Brassica carinata; cucumber plants (marrows, cucumbers, melons); fiber plants (cotton, flax, hemp, jute); vegetables (spinach, lettuce, asparagus, cabbages, carrots, eggplants, onions, pepper, tomatoes, potatoes, paprika, okra); plantation crops (bananas, fruit trees, rubber trees, tree nurseries), ornamentals (flowers, shrubs, broad-leaved trees and evergreens, such as conifers); as well as other plants such as vines, bushberries (such as blueberries), cane berries, cranberries, peppermint, rhubarb, spearmint, sugar cane and turf grasses including, but not limited to, cool-season turf grasses (for example, bluegrasses (Poa L.), such as Kentucky bluegrass (Poa pratensis L.), rough bluegrass (Poa trivialis L.), Canada bluegrass (Poa compressa L.) and annual bluegrass (Poa annua L.); bentgrasses (Agrostis L.), such as creeping bentgrass (Agrostis palustris Huds.), colonial bentgrass (Agrostis tenius Sibth.), velvet bentgrass (Agrostis canina L.) and redtop (Agrostis alba L.); fescues (Festuca L.), such as tall fescue (Festuca arundinacea Schreb.), meadow fescue (Festuca elatior L.) and fine fescues such as creeping red fescue (Festuca rubra L.), chewings fescue (Festuca rubra var. commutate Gaud.), sheep fescue (Festuca ovina L.) and hard fescue (Festuca longifolia); and ryegrasses (Lolium L.), such as perennial ryegrass (Lolium perenne L.) and annual (Italian) ryegrass (Lolium multiflorum Lam.) and warm-season turf grasses (for example, Bermudagrasses (Cynodon L. C. Rich), including hybrid and common Bermudagrass; Zoysiagrasses (Zoysia Willd.), St. Augustinegrass (Stenotaphrum secundatum (Walt.) Kuntze); and centipedegrass (Eremochloa ophiuroides (Munro.) Hack.)); vines; herbs; various fruits and vegetables of various botanical taxa such as Rosaceae spp. (for instance pip fruit such as apples and pears, but also stone fruit such as apricots, cherries, almonds and peaches, berry fruits such as strawberries), Ribesioidae spp., Juglandaceae spp., Betulaceae spp., Anacardiaceae spp., Fagaceae spp., Moraceae spp., Oleaceae spp., Actinidaceae spp., Lauraceae spp., Musaceae spp. (for instance banana trees and plantings), Rubiaceae spp. (for example, coffee), Theaceae spp., Malvaceae spp., Rutaceae spp. (for instance lemons, oranges and grapefruit), Solanaceae spp. (for instance tomatoes, potatoes, peppers, eggplant), Liliaceae spp., Asteraceae spp. (for instance lettuce, artichoke and chicory—including root chicory, endive or common chicory), Apiaceae spp. (for instance carrot, parsley, celery and celeriac), Cucurbitaceae spp. (for instance cucumber—including pickling cucumber, squash, watermelon, gourds and melons), Amaryllidaceae spp. (for instance onions and leek), Brassicaceae spp. (for instance white cabbage, red cabbage, broccoli, cauliflower, Brussel sprouts, pak choi, kohlrabi, radish, horseradish, cress, Chinese cabbage), Fabaceae spp. (for instance peanuts, peas and beans—such as climbing beans and broad beans), Chenopodiaceae spp. (for instance mangold, spinach beet, spinach, beetroots), Malvaceae (for instance okra), Asparagaceae (for instance asparagus); Grossulariaceae spp. (e.g., currents, gooseberries), Vitaceae spp. (e.g., grapes), Ericaceae spp. (e.g., blueberries, cranberries); horticultural and forest crops; ornamental plants; as well as genetically modified homologues of these crops.

### Fusarium graminearum

As described above, the crop disease Fusarium head blight (FHB) is primarily caused by the fungus Fusarium graminearum. Management of FHB currently requires an integrated approach of planting varieties with moderate or partial resistance, timely fungicide applications, and cultural practices such as crop rotation and tillage. However, there is a lack of complete host resistance available for F. graminearum. Hence, fungicides are often used to fight this disease. Widespread reliance on fungicide applications every year may lead to fungicide resistance, as variations in sensitivity have been found among F. graminearum isolates. Hence, integrated approaches including biocontrol would be useful for sustainable management of FHB.

As described herein, three endophytic fungi isolated from wheat were tested for their antagonism to F. graminearum. The experiments described herein i) phylogenetically characterize the endophytes using multiple loci, ii) assess the in-vitro competition against seven isolates of F. graminearum, and iii) assess the ability of the endophytes to increase seed mass and decrease mycotoxins in the presence of F. graminearum when applied to developing wheat heads in the greenhouse.

As illustrated herein, these three endophytes can be used to inhibit spread and mycotoxin production by Fusarium graminearum in agricultural crops. These endophytes were isolated from wheat microbiomes and shown to have antagonistic effects towards F. graminearum. Phylogenetic characterization showed that these endophytes were species Alternaria destruens, Fusarium commune, and Fusarium oxysporum.

### Alternaria destruens

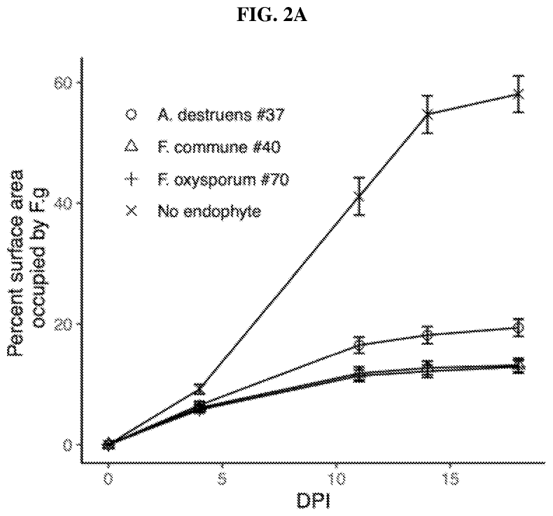

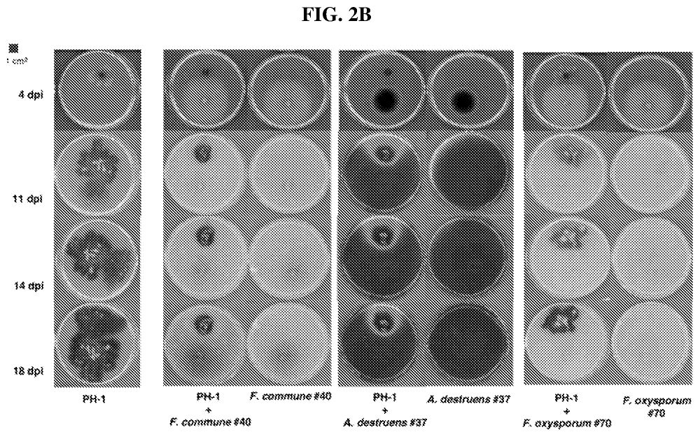

As illustrated herein in FIGS. 2A and 2B, discussed in Example 3 below, Alternaria destruens significantly restricted the area of Fusarium graminearum growth. For example, Alternaria destruens can restrict the area of Fusarium graminearum growth or reduce the area occupied by Fusarium graminearum by at least 5%, or at least 10%, or at least 15%, or at least 20%, or at least 25%, or at least 30%, or at least 35%, or at least 40%, or at least 45%, or at least 50%, or at least 55%, or at least 60%, or at least 70%, or at least 75%. In one experiment, the area occupied by Fusarium graminearum was about 41 mm² but when Alternaria destruens was used, the area occupied by Fusarium graminearum was reduced to about 18 mm². Hence, Alternaria destruens was effective for reducing the spread of Fusarium graminearum.

Sequence analysis and phylogenetic grouping of the Alternaria destruens isolate used in the experiments described herein (referred to as isolate #37) identified it as being an isolate of species Alternaria destruens. For example, sequence analysis showed that the ATPase gene (1194 bp) from Alternaria endophyte isolate #37 had a 99.83% match with 100% coverage compared to Alternaria destruens CBS 121454. The calmodulin gene (776 bp) had a one base pair mismatch and 100% coverage to Alternaria destruens CBS 121454 and Alternaria lini CBS 106.34. According to Woundenberg et al. (Woudenberg et al. 2015), the Alternaria destruens CBS 121454 and Alternaria lini CBS 106.34 species are synonyms of Alternaria alternata.

This ATPase gene from Alternaria endophyte #37 had the following sequence (SEQ ID NO:1).

```
      1 TACATCCTCT TCTACCGCGA CACCCGAACC AACCCTCACG
    41 CCGAGCAAAC CACCAAGAAG AAGGCCTGGT GGCAGTTCTG
    81 GAAGTCTGGC TCAGCTACCG CTGCCACTCC CATCCAGGAT
  121 GCCGGTGCCG TCCCCGACGA CTGTAAGTTT TATCATCCTG
  161 CTCACTCGAT TGCATGCACC TGCATCACAT AGCACTGCTG
  201 TTTGCGGCAG CGCTCAACGT ACCTCGCCAA TTCATCCTTT
  241 GTTGAGCTTT ACCTCGACAT TTGGTGGCTG GCATGGTCCG
  281 CGCTCAAGCT GCTCCCTGCT AGCGACGCGA TAGCGGCAGA
  321 AATGGTGGAG CCAATCATGC AATCCGGCTC CACCAAACTA
  361 CCCGCTTCTG CAGCATCCGA AATGAGCAAC ACGATCAAGA
  401 GGAATTTTGC TAACATGGAA TTGCAGACCT CAACACTGAG
  441 CTCCGAACTG GTCTCACCTC GTCCGACGTT GAGCAGCGTC
  481 GCAAGCGCTA TGGTTTCAAC GAAATCTCTT CTGAGAAGAC
  521 CAACCTTCTC AAGCAGTTCA TCGGTTACTT CACTGGTCCC
  561 ATTCTCTACG GTAAGCATCC CTGCACAAAC TTGTTTAGCG
  601 CCAAACTAAC GCATCATAGT CATGGAGCTC GCTGCTCTTC
  641 TCGCCGCTGG TCTTCAGGAT TGGGTCGATT TCGGTGTCAT
  681 CTGCGGTATC CTGTTGCTCA ACGCCATCGT CGGTTGGTAC
  721 CAGGAGAAAC AGGCTGCTGA TGTCGTCGCT TCGCTCAAGG
  761 GTGATATCGC CATGAAGGCC ACCGTCGTTC GTGACAACCA
  801 GCAACAGACC ATTCTCGCTC GTGAGCTTGT TCCCGGTGAC
  841 ATCGTCGTTA TTGAGGAGGG TCAATCCGTC CCCGGTGACG
  881 CCCGTCTTAT CTGCGGCTAC GACCACCCTG AGGACTTCGA
  921 CTTGTACATG AAGCTCAAGG CTGAGGACAA GTTCCACGAC
  961 GCTGACCCCG AGGACGAGAA GGATGACGAC GTCGATGAGG
1001 AGAAGTTCGA CGAGGAGAAC CCCATCACTC AGGGCCACCC
1041 TCTCGTTGCT TGCGATCAAT CGTCCATCAC CGGAGAGTCT
1081 CTCGCTGTCG ACAAGTACAT GGGAGAAGTC GCCTACTACA
1121 CCACTGGTTG CAAGCGCGGC AAGGCCTACG GTATCGTCAT
1161 CACCACTGCT AAGCACTCTT TCGTCGGTCG CACT
```

The Alternaria endophyte #37 also had a calmodulin gene (776 bp) with a one base pair mismatch and 100% coverage compared to Alternaria destruens CBS 121454 and Alternaria lini CBS 106.34. This calmodulin gene from Alternaria endophyte #37 had the following sequence (SEQ ID NO:2).

```
    1 CGTAAGTGCC CTCCCCATCC TCTGCCATGC CGCGCGGCTG
  41 CCTGGTAGCC CTGGGGGCCT GCGCAATCAC GAACATGCAG
  81 CTGACGACGT CGTGTTGTAG GACAAGGATG GCGATGGTCA
121 GTACTCTCCC TCCAAATTCC CTTCCACACA CACACTCTCT
161 CTCCCTCTCT GCCTTCAAAG CAGTGCCGCA TCTCCAGCCT
201 ACGCAATCGG CAGAGGGGCC CGGGCGAGGC TTGCTGGCTA
241 GGGGTCCAAA CCACCGCCCA CAGCTACAAC ACCACGACAT
281 CCACCCTACT CCATAGCAAG CACAACTGAC GACGATGCGC
321 CACAGGTCAA ATCACCACCA AGGAGCTAGG TACCGTCATG
361 CGCTCGCTCG GCCAAAATCC CAGCGAGTCT GAGCTCCAGG
401 ACATGATCAA CGAGGTCGAT GCCGACAACA ACGGCACCAT
441 TGACTTCCCA GGTGCGCCCC TTCATACCAG TCCAAAGTAC
481 CACAGCTAAC TTTTCCAGAA TTCCTTACCA TGATGGCCCG
521 CAAGATGAAG GACACCGACT CCGAGGAGGA GATCCGGGAA
561 GCCTTCAAGG TCTTCGACCG CGATAACAAC GGTTTCATCT
601 CCGCCGCCGA ACTGCGTCAC GTCATGACTT CTATTGGCGA
641 GAAATTGACC GATGACGAGG TCGACGAGAT GATCCGGGAG
681 GCTGACCAGG ACGGTGACGG CCGCATCGAC TGTAGGTTAC
721 AGCTGCCTAT ATCACGAGTG CGATGCTAAC ACACATCAGA
761 CAACGAGTTC GTCCA
```

The Alternaria endophyte #37 sequences can have sequence variability. For example, the Alternaria endophyte #37 sequences can have 1%, or 2%, or 3%, or 4%, or 5% sequence variability. In other words, Alternaria endophyte #37 sequences can have at least 95% sequence identity, or 96% sequence identity, or 97% sequence identity, or 98% sequence identity, or 99% sequence identity, or 99.5% sequence identity to the Alternaria endophyte #37 sequences described herein.

### Fusarium commune

As illustrated herein in FIGS. 2A and 2B, discussed in Example 3 below, Fusarium commune significantly restricted the area of Fusarium graminearum growth. For example, Fusarium commune can restrict the area of Fusarium graminearum growth or reduce the area occupied by Fusarium graminearum by at least 10%, or at least 15%, or at least 20%, or at least 25%, or at least 30%, or at least 35%, or at least 40%, or at least 45%, or at least 50%, or at least 55%, or at least 60%, or at least 70%, or at least 75%. In one experiment, Fusarium commune restricted the area of Fusarium graminearum growth by about 36% to 42% at 14 days after inoculation of the Fusarium graminearum with the Fusarium commune. As illustrated herein, in one experiment the area occupied by Fusarium graminearum was about 41 mm² but when Fusarium commune was used, the area occupied by Fusarium graminearum was reduced to 12-13 mm². Hence, Fusarium commune was effective for reducing the spread of Fusarium graminearum.

Sequence analysis and phylogenetic grouping of the Fusarium commune endophyte isolate identified it as being an isolate of Fusarium commune NRRL 28387. This Fusarium commune isolate is also related to NRRL 13816 and NRRL 28058. For example, the Fusarium commune endophyte isolate number 40 has an RPB1 sequence that exactly matched (100% identical, 100% coverage) the RPB1 from Fusarium commune NRRL 28387. The Fusarium commune NRRL 28387 strain can be obtained from the NRRL Agricultural Research Service Culture Collection online catalog (see website at nrrl.ncaur.usda.gov/cgi-bin/usda/fungi/report.html?nrrlcodes=28387). Additionally, the EF1-α sequence had an exact match (100% identical, 100% overlap) to the EF1-α sequence of two Fusarium commune isolates (NRRL 28058 and NRRL 13816).

The Fusarium endophyte #40 isolate had the following RPB1 gene sequence (SEQ ID NO:3), which had 100% sequence identity (with 100% coverage) compared to a RPB1 from Fusarium commune NRRL 28387.

```
      1 TTTTCCTCAC AAAGGAGCAA ATCATGAACT GTATGCTCTG
    41 GGTGCCTAAC TGGGACGGTG TCATTCCTCA ACCCGCTATC
    81 TATAAGCCTC GTCCTCGGTG GACTGGTAAG CAGCTTATCA
  121 GCATGGTTAT CCCTAAGGAG GTTAGCCTGT TCAACGGTAC
  161 GGATTCTGGT GAAAACGCCC CTCTTAAGGA CGAGGGTCTT
  201 CTGATCCAAG CTGGCCAACT GATGTATGGT CTTTTGACTA
  241 AGAAGAACAT TGGTGCTGCT GCGGGCGGTA TTGTGCATAT
  281 CAGCTACAAC GAACTTGGCC CCGAAGGTGC GATGGCTTTC
  321 TTGAACGGTG TCCAGCAGGT TGTCACCTAC TGGCTTCTCA
  361 ACAATGGCCA TAGCATTGGT ATTGGTGATA CAATTCCCGA
  401 TGCGGCGACC ATTGCTAAAG TTCAGGTACA TATTGATGAG
  441 GAAAAGGCTG AAGTTGCTCG CTTGACAGCA ATGGCCACAG
  481 CGAATGAGCT TGAGGCCCTA CCTGGTATGA ACGTTCGTGC
  521 AACCTTCGAA AACAAGGTCT CCATGGCTCT GAACCAGGCC
  561 CGTGATAAGG CTGGTACCAC AACACAGAAG AGTTTGAAGG
  601 ATTCAAACAA CGCTGTCACC ATGGCTTCCT CAGGTTCCAA
  641 GGGTTCATCT ATCAATATTT CTCAAATGAC TGCGCTTGTC
  681 GGTCAGCAAA TTGTCGAAGG CAAGCGTATT CCTTTTGGTT
  721 TCAAGTATCG CACATTACCT CACTTCACCA AGGACGATTA
  761 CTCACCTGAG GCCCGTGGCT TCGTCGAGAA CTCTTACCTC
  801 CGTGGTCTCA CTCCCAGCGA GTTTTTCTTC CACGCCATGG
  841 CTGGTCGAGA AGGTCTCATT GATACTGCAG TCAAGACTGC
  881 CGAAACAGGT TATATCCAGC GACGATTGGT CAAGGCTCTG
  921 GAAGATCTTT CTGCCCGTTA CGATGGAACT GTCCGAAACT
  961 CTCTGGGAGA CATTGTTCAG TTCCTCTATG GTGAAGACGG
1001 TCTCGATGCC ATGATTATTG AGAAACAGAA GTTGGGTATC
1041 CTCAATATGT CAAACTCGGC ATTTGAAAAG AAGTATCGTC
1081 TGGATCTTGC CAACCCCCCG GACTGGTTTA AGCACGACTA
1121 CGAATTCGGT AATGAATTGA CTGGTGACAA GGAATCTATG
1161 GAGTATCTCG ATCAAGAATG GGAAAAGTTG TTGGCTGATC
1201 GCAGACAAAT CCGACAGATC AACAAGGCCA AGGGTAACGA
1241 GGAAATGATG CAACTGCCCC TCAACATCAC TCGCATCATC
1281 GAGTCTGCTA AGCGAGTCTT TAATGTCAAG GCTAATGACC
1321 GAAGCAACTT GCGACCTTCG GAAGTTATTC CAGCTGTGCA
1361 AAACTTGTTG GATAGCATGA AGATTGTTCG TGGTACTGAT
1401 GAAATCTCGA TTGAAGCTGA CGCAAATGCA TCCATTCTCT
1441 TCAAGGCCTT GCTTCGCTCT CGCCTGGCCT TCAAGGAGGT
1481 GGTCAAGGAG CACCGGTTGA ACAAATTAGC TTTCGACCAT
1521 ATTCTGGGTG AACTCCAGAA TAGATGGGAT CGCGCATTTG
1601 TCA
```

The Fusarium endophyte #40 isolate had the following RPB2 gene sequence (SEQ ID NO:4), which had 99.76% sequence identity to a RPB2 from two Fusarium commune isolates (NRRL 13816 and NRRL 28058).

```
      1 TTGACAGATA TACCTTTGCT TCGACTCTTT CACATTTGCG
    41 TCGAACCAAT ACTCCTATTG GACGAGATGG TAAATTGGCC
    81 AAGCCTCGAC AGCTTCACAA TACTCACTGG GGTTTGGTGT
  121 GTCCCGCCGA AACGCCTGAG GGTCAAGCTT GTGGTCTGGT
  161 CAAAAACTTG TCTCTGATGT GTTATGTCAG TGTCGGCTCT
  201 CCAGCCGAGC CTCTCATTGA ATTCATGATC AACAGAGGTA
  241 TGGAAGTTGT TGAGGAGTAC GAGCCGACAA GATATCCCCA
  281 CGCTACAAAG ATTTTCGTCA ACGGTAGCTG GGTTGGTGTT
  321 CACGCCGACC CCAAGCATCT CGTGAATCAG GTTTTGGACA
  361 CAAGACGAAA GTCGTACGTT CAGTTCGAAG TATCACTTGT
  401 TCGTGATATT CGAGACCGTG AATTCAAGAT TTTCTCAGAC
  441 GCTGGTCGTG TCATGAGACC CGTCTTTACA GTCCATCAGG
  481 AGGATGACTA TGAGAACAAC ATCACCAAGG GACAACTAGT
  521 GTTGACAAAG GAACATGTCA ATAGGCTAGC CCAAGAGCAG
  561 GCAGAGCCAC CTGCCAACCC CGCGGACAAG TTTGGATGGG
  601 ATGGCTTGAT TCGCGAAGGA GCTGTCGAGT ATCTCGACGC
  641 TGAGGAAGAA GAGACAGCCA TGATTTGCAT GACGCCAGAG
  681 GATCTCGAAC TTTACCGTGA GCAAAAGAAT GATGAAGCTA
  721 CACTCACGGA AGAAGAGAAA CGGGCCAAGG CAGAGGCAGA
  761 GAAGAGGGAA CAAGAGGAGG ACCGCAACAA GCGATTGAAG
  801 ACAAAGGTCA ACCCCACAAC TCACATGTAC ACACATTGTG
  841 AGATTCACCC CAGTATGATT CTCGGTATCT GTGCCAGTAT
  881 CATTCCTTTC CCCGATCACA ACCAGGTATG TAGTCCCTTT
  921 GATCACAACA ACCTCAACNN NNNNNNNNNN NNNNNNNNNN
  961 NNNNNNNNNN NNNNNNNNNN NNNNNNNNNN NCAATATTCT
1001 CTACTACCCT CAAAAGCCTC TCGCCACTAC CCGATCCATG
1041 GAGTTCCTCA AGTTCCGTGA ATTGCCAGCT GGTCAAAATG
1081 CCATTGTCGC AATTGCTTGC TACTCAGGTT ATAACCAGGA
1121 AGATTCCGTC ATTATGAACC AGAGTAGTAT TGATCGAGGT
1161 CTGTTCCGAA GTCTGTTCTT CCGATCGTAC TCAGACCAGG
1201 AGAAGAAGGT CGGCCTCAAC TACACTGAGA TCTTTGAGAA
1241 GCCTTTCCAG CAGACAACAC TCCGAATGAA GCATGGAACA
1281 TACGACAAGC TTGACGAGGA TGGTATCGTG GCTCCTGGTG
1321 TCCGTGTGTC TGGTGAAGAT ATCATTATCG GCAAGACTGC
1361 ACCCATCGAC CAAGAAAACC AGGACCTTGG CACAAGGACT
1401 CAATCGCACC AGCGTCGTGA TATCTCGACA CCACTGCGAA
1441 GTACCGAGAA CGGTATCGTT GATCAGGTCA TTCTGACAGT
1481 CAACGCCGAT AACGTCAAGT ACGTCAAGGT TCGAGTACGA
1521 ACAACCAAGA TTCCTCAAAT CGGTGACAAG TTTGCTTCTC
1561 GTCACGGTCA AAAGGGTACA ATCGGTGTTA CATATCGACA
1601 GGAGGATATG CCTTTCAGCC GAGAAGGTCT CACCCCCGAT
1641 ATCATTATCA ACCCTCACGC CATTCCATCG CGAATGACAA
1681 TTGCCCATTT GATTGAGTGT CTCCTTAGCA AGGTTTCAAC
1721 GCTGGAAGGT ATGGAGGGTG ACGCCACACC GTTCACTGAT
1761 GTCACAGTCG ATTCAGTCTC AGAACTTCTG AGGAAGCACG
1801 GTTACCAATC TCGAGGTTTC GAGGTCATGT ACAATGGTCA
1841 CACTGGACGA AAGCTCCGTG CCCAGGTGTT CTTCGGACCT
1881 ACCTACTAC
```

The Fusarium endophyte #40 isolate had the following elongation factor 1 alpha (EF1a) gene sequence (SEQ ID NO:5), which had 100% sequence identity (and 100% coverage) to an EF1-α sequence of two Fusarium commune isolates (NRRL 28058 and NRRL 13816).

```
    1 GACTCACCTT AACGTCGTCG TCATCGGCCA CGTCGACTCT
  41 GGCAAGTCGA CCACTGTGAG TACTCCCCTT GGACGATGAG
  81 CTTATCTGCC ATCGTTAATC CCGACCAAGA CCTGGCGGGG
121 TATTTCTCAA AGGCAATATG CTGATATCGT TTCACAGACC
161 GGTCACTTGA TCTACCAGTG CGGTGGTATC GACAAGCGAA
201 CCATCGAGAA GTTCGAGAAG GTTAGTCACT TTCCCTTCGA
241 TCGCGCGTCC TCTGCCCATC GATTTCCCCT ACGACTCGAA
281 ACCTGCCCGC TACCCCGCTC GAGACCAAAA ATTTTGCGAT
321 ATGACCGTAA TTTTTTTTGG TGGGGCATTT ACCCCGCCAC
361 TCGAGCGACG GGCGCGTTTG CCCTCCTCCC ATTTCCACAA
401 CCTCAATGAG CGCATCGTCA CGTGTCACGC AGTCACTAAC
441 CATTCAATAA TAGGAAGCCG CTGAGCTCGG TAAGGGTTCC
481 TTCAAGTACG CCTGGGTTCT TGACAAGCTC AAGGCCGAGC
521 GTGAGCGTGG TATCACCATC GATATTGCTC TCTGGAAGTT
561 CGAGACTCCT CGCTACTATG TCACCGTCAT TGGTATGTTG
601 TCGCTCATGC TTCATTCTAC TTCTCTTCGT ACTGACATAT
641 CACTCAGACG CTCCCGGTCA CCGTGATTTC ATCAAGAA
```

Accordingly, the Fusarium commune endophyte isolate #40 described herein is of species Fusarium commune and can be identified by sequence or obtained from the NRRL.

The Fusarium endophyte #40 isolate sequences can have sequence variability. For example, the Fusarium endophyte #40 isolate sequences can have 1%, or 2%, or 3%, or 4%, or 5% sequence variability. In other words, Fusarium endophyte #40 isolate sequences can have at least 95% sequence identity, or 96% sequence identity, or 97% sequence identity, or 98% sequence identity, or 99% sequence identity, or 99.5% sequence identity to the Fusarium endophyte #40 isolate sequences described herein.

### Fusarium oxysporum

As illustrated herein in FIGS. 2A and 2B, discussed in Example 3 below, Fusarium oxysporum significantly restricted the area of Fusarium graminearum growth. For example, Fusarium oxysporum can restrict the area of Fusarium graminearum growth or reduce the area occupied by Fusarium graminearum by at least 10%, or at least 15%, or at least 20%, or at least 25%, or at least 30%, or at least 35%, or at least 40%, or at least 45%, or at least 50%, or at least 55%, or at least 60%, or at least 70%, or at least 75%. In one experiment, Fusarium oxysporum restricted the area of Fusarium graminearum growth by about 36% to 42% at 14 days after inoculation of the Fusarium graminearum with the Fusarium oxysporum. As illustrated herein, in one experiment the area occupied by Fusarium graminearum was about 41 mm² but when Fusarium oxysporum was used, the area occupied by Fusarium graminearum was reduced to 12-13 mm². Hence, Fusarium oxysporum was effective for reducing the spread of Fusarium graminearum.

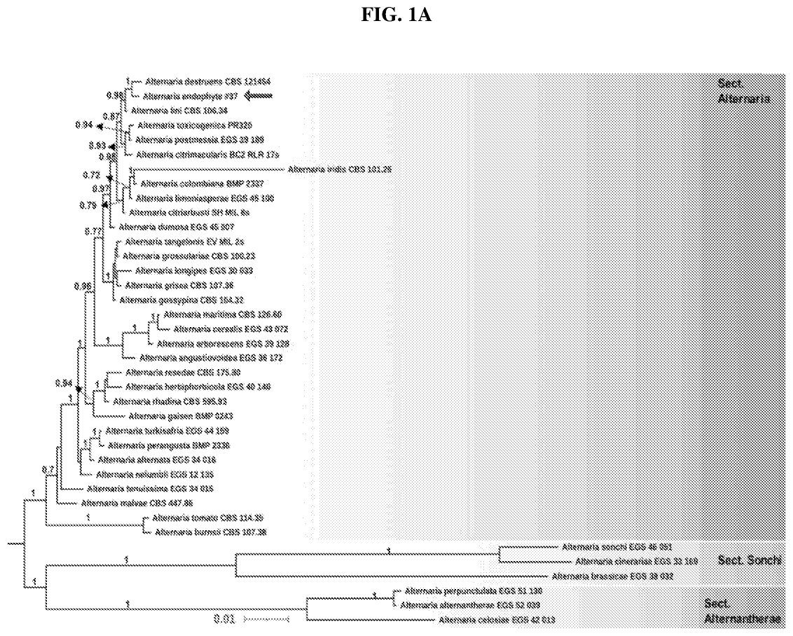

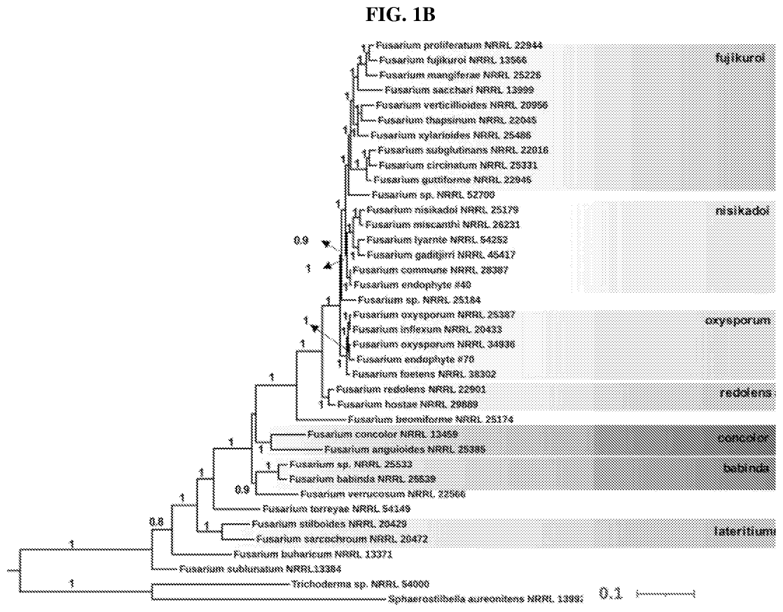

Sequence analysis and phylogenetic grouping of the Fusarium oxysporum isolate used in the experiments described herein (referred to as isolate #70) identified it as being an isolate of species Fusarium oxysporum. RPB1 and RPB2 sequences of the #70 isolate grouped it with Fusarium oxysporum NRRL 34936 in a well-supported (posterior probability=1.0) monophyletic group (FIG. 1B). In terms of sequence identity, Fusarium #70 RBP2 sequence (1887 bp) had a 99.75% match with 97.35% overlap to Fusarium oxysporum NRRL 1943. The RPB1 sequence (1470 bp) had a 99.66% match with a 100% coverage to Fusarium oxysporum NRRL 20433. Additionally, the EF1-α sequence (684 bp) had a 99.27% match and 99.56% overlap with Fusarium oxysporum NRRL 1943.

The Fusarium endophyte #70 isolate had the following RPB1 gene sequence (SEQ ID NO:6), which had 99.66% sequence identity (and 100% coverage) to a RPB1 sequence from Fusarium oxysporum NRRL 20433.

```
      1 TTTTCCTCAC AAAGGAGCAA ATCATGAACT GTATGCTCTG
    41 GGTGCCCAAC TGGGACGGTG TCATTCCTCA ACCCGCTATC
    81 TATAAGCCTC GTCCTCGGTG GACTGGTAAG CAGCTTATCA
  121 GCATGGTTAT CCCTAAGGAG GTTAGCCTGT TCAACGGTAC
  161 GGATTCTGGT GAAAACGCCC CTCTTAAGGA CGAGGGTCTT
  201 CTGATCCAAG CCGGCCAACT GATGTATGGT CTTTTAACTA
  241 AGAAGAACAT TGGTGCTGCT GCGGGTGGTA TTGTGCATAT
  281 CAGCTACAAC GAACTTGGCC CCGAAGGTGC GATGGCTTTC
  321 TTAAACGGTG TCCAGCAGGT TGTCACCTAC TGGCTTCTCA
  361 ACAATGGTCA TAGCATTGGT ATTGGTGATA CAATTCCCGA
  401 TGCGGCGACC ATTGCTAAAG TTCAGGTACA TATTGATGAG
  441 GAAAAGGCTG AAGTTGCCCG CTTGACAGCA ATGGCCACAG
  481 CGAATGAGCT TGAGGCCCTA CCTGGTATGA ACGTTCGTGC
  521 AACCTTCGAA AACAAAGTCT CCATGGCTCT GAACCAGGCC
  561 CGTGATAAGG CTGGTACCAC AACACAGAAG AGTTTGAAGG
  601 ATTCAAACAA CGCTGTCACC ATGGCTTCCT CAGGTTCCAA
  641 GGGTTCATCT ATCAATATTT CTCAAATGAC TGCGCTTGTC
  681 GGTCAGCAAA TTGTCGAAGG CAAGCGTATT CCTTTTGGTT
  721 TCAAGTATCG CACATTACCT CACTTCACCA AGGACGATTA
  761 CTCACCTGAG GCCCGTGGCT TCGTCGAGAA CTCTTACCTC
  801 CGTGGTCTCA CTCCTAGCGA ATTTTTCTTC CACGCCATGG
  841 CTGGTCGAGA AGGTCTCATT GATACTGCAG TCAAGACTGC
  881 CGAAACAGGT TATATCCAGC GACGATTGGT TAAGGCTCTG
  921 GAAGATCTTT CTGCCCGTTA CGATGGAACT GTCCGAAACT
  961 CTCTGGGAGA CATTGTTCAG TTCCTCTATG GTGAAGACGG
1001 TCTTGATGCC ATGATTATTG AGAAACAGAA GTTGGGTATC
1041 CTCAATATGT CAAACTCGGC ATTTGAAAAG AAGTATCGTC
1081 TGGATCTTGC CAACCCCCCG GACTGGTTTA AGCACGACTA
1121 CGAATTCGGT AACGAATTGA CTGGTGACAA GGAATCTATG
1161 GAGTATCTCG ATCAAGAATG GGAAAAGTTG TTGGCTGATC
1201 GCAGACAAGT CCGACAGATC AACAAGGCCA AGGGTAACGA
1241 GGAAATGATG CAACTGCCCC TCAACATCAC TCGCATCATC
1281 GAGTCTGCTA AGCGAGTCTT TAATGTCAAG GCTAATGACC
1321 GAAGCAACTT GCGACCGTCG GAAGTTATTC CAGCTGTGCA
1361 AAACTTGTTG GATAGCATGA AGATTGTTCG TGGTACTGAT
1401 GAAATCTCGA TTGAAGCTGA CGCAAATGCA TCATTTCTCT
1441 TCAAGGCCTT GCTTCGCTCT CGCCTGGCCT
```

The Fusarium endophyte #70 isolate had the following RPB2 gene sequence (SEQ ID NO:7), which had 99.75% sequence identity to a RPB2 from to Fusarium oxysporum NRRL 1943.

```
      1 TGCTTCTACT CTTTCACATT TGCGTCGAAC CAATACTCCC
    41 ATCGGACGAG ATGGTAAATT GGCCAAGCCT CGACAGCTTC
    81 ACAACACTCA CTGGGGTTTG GTGTGTCCTG CCGAAACACC
  121 TGAGGGTCAA GCTTGTGGTC TGGTCAAAAA CTTGTCTCTA
  161 ATGTGTTACG TCAGTGTCGG CTCTCCAGCC GATCCTCTGA
  201 TTGAATTCAT GATCAACAGA GGCATGGAAG TCGTTGAGGA
  241 GTACGAGCCG ACAAGATACC CCCACGCTAC AAAGATTTTC
  281 GTCAACGGTA GCTGGGTTGG TGTTCATGCC GACCCCAAGC
  321 ATCTCGTGAA TCAGGTCTTG GACACAAGAC GAAAGTCTTA
  361 CGTGCAGTTC GAAGTATCAC TTGTTCGTGA TATCCGAGAC
  401 CGTGAATTCA AGATTTTTTC AGACGCTGGC CGTGTCATGA
  441 GACCCGTCTT TACAGTTCAT CAGGAGGATG ACTATGAGAA
  481 CAACATCACC AAGGGACAAC TAGTGTTGAC AAAGGACCAT
  521 GTCAATAGGC TAGCCCAAGA ACAGGCAGAG CCTCCTGCCA
  561 ACCCAGCGGA CAAGTTTGGA TGGGATGGCT TGATCCGCGA
  601 AGGAGCTGTC GAGTATCTCG ATGCTGAGGA AGAAGAGACA
  641 GCCATGATTT GCATGACGCC AGAGGATCTC GAACTTTACC
  681 GTGAGCAAAA GAATGATGAA GCTACACTCA CAGAAGAAGA
  721 GAAACGGGCC AAGCAAGAGG CAGAGAAGAG AGAACAAGAG
  761 GAGGACCGCA ACAAGCGATT GAAGACAAAG GTGAACCCCA
  801 CAACTCACAT GTACACACAT TGTGAGATTC ACCCCAGTAT
  841 GATTCTCGGT ATCTGTGCCA GTATCATTCC TTTCCCCGAT
  881 CACAACCAGG TATGTNNNNN NNNNNNNNNN NNNNNNNNNN
  921 NNNNNNNNNN NNNNNNNNNN NNNNNNNNNN NNNNNNNNNN
  961 NNNNNNNNNN NNNNNNNNNN NNNNNNNNNN NNNNNNNNNN
1001 NNNNNNNNNN NNNNNNNNNN NNNNNNNNNN NNNNNNNNNN
1041 NNNNNNNNNN NNNNNNNNNN NNNNNNNNNN NNNNNNNNNN
1081 NNNNNNNNNN NNNNNNNNNN NNNNNNNNNN NNNNNNNNNN
1121 NNNNNNAGTA GTATTGATCG AGGTTTGTTC CGAAGTCTGT
1161 TCTTCCGATC GTACTCAGAT CAGGAGAAGA AGGTTGGTCT
1121 CAACTACACT GAGATCTTTG AGAAACCTTT CCAGCAGACA
1201 ACGCTTCGAA TGAAGCATGG AACATACGAC AAGCTTGATG
1241 AAGATGGTAT CGTGGCTCCT GGTGTCCGTG TGTCAGGTGA
1281 AGATATCATT ATCGGCAAGA CTGCACCCAT CGACCAAGAA
1321 AACCAGGACC TTGGCACAAG AACTCAATCG CACCAACGTC
1361 GTGATATCTC GACACCACTG CGAAGTACTG AGAACGGTAT
1401 CGTTGATCAA GTCATTCTGA CAGTCAACGC CGATAACGTC
1441 AAGTACGTCA AGGTCCGAGT ACGAACAACC AAGATTCCTC
1481 AAATTGGTGA CAAGTTTGCT TCTCGTCACG GTCAAAAGGG
1521 TACAATCGGT GTTACATATC GACAGGAGGA TATGCCTTTC
1561 AGCCGAGAAG GTCTTACTCC CGATATCATT ATCAACCCTC
1601 ACGCCATTCC ATCGCGAATG ACAATTGCCC ATTTGATTGA
1641 GTGTCTTCTT AGCAAGGTTT CAACGCTGGA AGGTATGGAG
1681 GGTGACGCCA CACCGTTCAC TGATGTCACA GTCGATTCAG
1721 TCTCAGAACT TCTGAGGAAG CACGGTTACC AATCTCGAGG
1761 TTTCGAGGTC ATGTACAACG GTCACACTGG ACGAAAGCTC
1801 CGTGCCCAGG
```

The Fusarium endophyte #70 isolate also had the following translation elongation factor 1 alpha (TEF1a) gene sequence (SEQ ID NO:8), which had 99.27% sequence identity and 99.56% overlap with Fusarium oxysporum NRRL 1943.

```
    1 AGACAAGACT CACCTTAACG TCGTCGTCAT CGGCCACGTC
  41 GACTCTGGCA AGTCGACCAC TGTGAGTACT CTCCTTGACA
  81 ATGAGCTTAT CTGCCATCGT CAATCCCGAC CAAGACCTGG
121 CGGGGTATTT CTCAAAGTCA ACATACTGAC ATCGTTTCAC
161 AGACCGGTCA CTTGATCTAC CAGTGCGGTG GTATCGACAA
201 GCGAACCATC GAGAAGTTCG AGAAGGTTAG TTACTTTCCC
241 TTCGATCGCG CGTCTTTTGC CCATCGATTT CCCCTACGAC
281 TCGAAACGTG CCCGCTACCC CTCTCGAGAC CAAAAATTTT
321 GCAATATGAC CGTAATTTTT TTGGTGGGGC ATTTACCCCG
361 CCCCTCGGGT GCCGGGCGCG TTTGCCCTCT TACCATTCTC
401 ACAACCTCAA TGAGCGCATC GTCACGTGTC AAGCAGTCAC
441 TAACCATTCA ACAATAGGAA GCCGCTGAGC TCGGTAAGGG
481 TTCCTTCAAG TACGCCTGGG TTCTTGACAA GCTCAAGGCC
521 GAGCGTGAGC GTGGTATCAC CATCGATATT GCTCTCTGGA
561 AGTTCGAGAC TCCTCGCTAC TATGTCACCG TCATTGGTAT
601 GTTGTCGCTC ATGCTTCATT CTACTTCTCT TCGTACTAAC
641 ACATCACTCA GACGCTCCCG GTCACCGTGA TTTCATCAAG
661 AACATGA
```

Accordingly, the Fusarium oxysporum endophyte isolate #70 described herein is of species Fusarium oxysporum.

The Fusarium endophyte #70 isolate sequences can have sequence variability. For example, the Fusarium endophyte #70 isolate sequences can have 1%, or 2%, or 3%, or 4%, or 5% sequence variability. In other words, Fusarium endophyte #70 isolate sequences can have at least 95% sequence identity, or 96% sequence identity, or 97% sequence identity, or 98% sequence identity, or 99% sequence identity, or 99.5% sequence identity to the Fusarium endophyte #70 isolate sequences described herein.

As discussed above the Fusarium endophyte isolate #70 is of species Fusarium oxysporum. The Fusarium oxysporum strains, including Fusarium oxysporum NRRL 34936, NRRL 1943 and NRRL 20433 can be obtained from the NRRL Agricultural Research Service Culture Collection online catalog. For example, the Fusarium oxysporum NRRL 34936 strain can be obtained from the NRRL catalog as provided by website nrrl.ncaur.usda.gov/cgi-bin/usda/fungi/report.html?nrrlcodes=34936 (see also Genbank JABFES000000000.1). The Fusarium oxysporum NRRL 1943 strain can be obtained from the NRRL catalog as provided by website nrrl.ncaur.usda.gov/cgi-bin/usda/fungi/report.html?nrrlcodes=1943. The Fusarium oxysporum NRRL 20433 strain can be obtained from the NRRL catalog as provided by website nrrl.ncaur.usda.gov/cgi-bin/usda/fungi/report.html?nrrlcodes=20433.

### Mycotoxins

The compositions and methods described herein can inhibit the spread of Fusarium graminearum, can reduce or inhibit mycotoxins in agricultural products. Mycotoxins are toxic fungal metabolites, often found in agricultural products that are characterized by their ability to cause health problems for humans and vertebrates. Mycotoxins include compounds such as aflatoxins, ochratoxins, patulin, fumonisins, zearalenones, and trichothecenes. They are produced for example by different Fusarium, Aspergillus, Penicillium and Alternaria species.

Mycotoxins can cause immunosuppressive, carcinogenic, cytotoxic and teratogenic effects in humans and animals who consume contaminated grains and nuts. For example, deoxynivalenol, a mycotoxin common in wheat and barley in the U.S. Midwest, is produced by several Fusarium species on grain crops. Deoxynivalenol has immunosuppressive effects on humans and induces feed refusal in animals. The FDA suggests levels of less than 15 ppm deoxynivalenol in finished products for human consumption.

Aflatoxins are toxins produced by Aspergillus species and other fungal taxa that grow on several crops, in particular on maize or corn before and after harvest of the crop as well as during storage. The biosynthesis of aflatoxins involves a complex polyketide pathway starting with acetate and malonate. One important intermediate is sterigmatocystin and O-methylsterigmatocystin which are direct precursors of aflatoxins. Important producers of aflatoxins are Aspergillus flavus, most strains of Aspergillus parasiticus, Aspergillus nomius, Aspergillus bombycis, Aspergillus pseudotamarii, Aspergillus ochraceoroseus, Aspergillus rambelli, Emericella astellata, Emericella venezuelensis, Bipolaris spp., Chaetomium spp., Farrowia spp., and Monocillium spp., in particular Aspergillus flavus and Aspergillus parasiticus (Plant Breeding (1999), 118, pp 1-16). There are also additional Aspergillus species known. The group of aflatoxins consists of more than 20 different toxins, for example, aflatoxin B1, B2, G1 and G2, cyclopiazonic acid (CPA).

Ochratoxins are mycotoxins produced by some Aspergillus species and Penicilium species, like A. ochraceus, A. carbonarius or P. viridicatum, Examples for Ochratoxins are ochratoxin A, B, and C. Ochratoxin A is the most prevalent and relevant fungal toxin of this group.

Fumonisins are toxins produced by Fusarium species that grow on several crops, mainly corn, before and after harvest of the crop as well as during storage. The diseases, Fusarium kernel, ear and stalk rot of corn, is caused by Fusarium verticillioides, Fusarium subglutinans, Fusarium moniliforme, and Fusarium proliferatum. The main mycotoxins of these species are the fumonisins, of which more than ten chemical forms have been isolated. Examples for fumonisins are FB I, FB2 and FB3. In addition, the above-mentioned Fusarium species of corn can also produce the mycotoxins moniliformin and beauvericin. In particular, Fusarium verticillioides is mentioned as an important pathogen of corn, and this Fusarium species produces the main mycotoxin fumonisins of the B-type. Trichothecenes are those mycotoxins of primary concern which can be found in Fusarium diseases of small grain cereals like wheat, barley, rye, triticale, rice, sorghum and oat. They are sesquiterpene epoxide mycotoxins produced by species of Fusarium, Trichothecium, and Myrothecium and act as potent inhibitors of eukaryotic protein synthesis. Some of these trichothecene producing Fusarium species also infect corn or maize.

Examples of trichothecene mycotoxins include T-2 toxin, HT-2 toxin, isotrichodermol, diacetoxyscirpenol (DAS), 3-deacetylcalonectrin, 3,15-dideacetylcalonectrin, scirpentriol, neosolaniol; 15-acetyldeoxynivalenol, 3-acetyldeoxynivalenol, nivalenol, 4-acetylnivalenol (fusarenone-X), 4,15-diacetylnivalenol, 4,7,15-acetylnivalenol, and deoxynivalenol (“DON”) and their various acetylated derivatives. The most common trichothecene in Fusarium head blight is deoxynivalenol produced for example by Fusarium graminearum and Fusarium culmorum.

Another mycotoxin mainly produced by F. culmorum, F. graminearum and F. cerealis is zearalenone, a phenolic resorcyclic acid lactone that is primarily an estrogenic fungal metabolite.

Fusarium species that produce mycotoxins, such as fumonisins and trichothecenes, include F. acuminatum, F. crookwellense, F., verticillioides, F. culmorum, F. avenaceum, F. equiseti, F. moniliforme, F. graminearum (Gibberella zeae), F. lateritium, F. poae, F. sambucinum (G. pulicaris), F. proliferatum, F. subglutinans, F. sporotrichioides and other Fusarium species.

In contrast, the species Microdochium nivale also a member of the so-called Fusarium complex is known to not produce any mycotoxins. Both acute and chronic mycotoxicoses in farm animals and in humans have been associated with consumption of wheat, rye, barley, oats, rice and maize contaminated with Fusarium species that produce trichothecene mycotoxins. Experiments with chemically pure trichothecenes at low dosage levels have reproduced many of the features observed in moldy grain toxicoses in animals, including anemia and immunosuppression, hemorrhage, emesis and feed refusal. Historical and epidemiological data from human populations indicate an association between certain disease epidemics and consumption of grain infected with Fusarium species that produce trichothecenes. In particular, outbreaks of a fatal disease known as alimentary toxic aleukia, which has occurred in Russia since the nineteenth century, have been associated with consumption of over-wintered grains contaminated with Fusarium species that produce the trichothecene T-2 toxin. In Japan, outbreaks of a similar disease called akakabi-byo or red mold disease have been associated with grain infected with Fusarium species that produce the trichothecene, DON. Trichothecenes were detected in the toxic grain samples responsible for recent human disease outbreaks in India and Japan. There exists, therefore, a need for agricultural methods for preventing, and crops having reduced levels of, mycotoxin contamination. Further, mycotoxin-producing Fusarium species are destructive pathogens and attack a wide range of plant species. The acute phytotoxicity of mycotoxins and their occurrence in plant tissues also suggests that these mycotoxins play a role in the pathogenesis of Fusarium on plants. This implies that mycotoxins play a role in disease and, therefore, reducing their toxicity to the plant may also prevent or reduce disease in the plant. Further, reduction in disease levels may have the additional benefit of reducing mycotoxin contamination on the plant and particularly in grain where the plant is a cereal plant.

### Compositions

Compositions described herein can include at least one endophyte, part, or propagule thereof. For example, the endophytes can be whole, shredded, ground up, granulated, or mixtures of endophyte tissues. However, in some cases, the endophyte spores or conidia are more conveniently used in the compositions described herein.

The composition can contain varying amounts of the endophyte components, endophyte spores, or endophyte conidia described herein. For example, the spores or conidia can be present in liquid compositions at concentrations per milliliter of about 1×10² to about 1×10¹², or about 1×10³ to about 1×10¹¹, or about 1×10³ to about 1×10¹⁰, or about 1×10³ to about 1×10⁹, or about 1×10³ to about 1×10⁸, or about 1×10³ to about 1×10⁷, or about 1×10⁴ to about 1×10⁷, or about 1×10⁴ to about 1×10⁹, or about 1×10⁴ to about 1×10¹⁰.

In dry compositions, the spores or conidia can be present at weight/weight concentrations of about 0.1 μg/g to about 1000 μg/g, or about 1 μg/g to about 800 μg/g, or about 3 μg/g to about 600 μg/g, or about 5 μg/g to about 500 μg/g, or about 5 μg/g to about 300 μg/g. The compositions can therefore be dry compositions or liquid compositions.

The compositions can also include additional components such as a carrier, solvent, surfactant, an additional active ingredient, or a combination thereof. In some instances, the endophytes, parts, or propagules thereof are dissolved in a carrier to form a dry or liquid composition with a known concentration of at least one component. The carrier can be an aqueous carrier, that can also contain an alcohol, glycerol, emulsifier, dispersing agent, thickening agent, a surfactant, a clay, a polymer, a colorant, a wetting agent of ionic or non-ionic type, a natural or regenerated mineral substance, a dispersant, a wetting agent, a tackifier, a thickener, a binder, or a mixture of such carriers. For example, the compositions can contain polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives thereof. The presence of at least one surfactant can be included when the endophytes, parts, or propagules thereof are water-insoluble and when the composition for application is water. For example, surfactant content can be about 5% to 40% by weight of the composition.

Optionally, additional components may also be included, e.g., protective colloids, adhesives, thickeners, thixotropic agents, penetration agents, stabilizers, sequestering agents.

The compositions can also include other ingredients. For example, bactericidal compounds can be employed. In addition, different types of the endophytes, parts, or propagules thereof described herein can be used together in a composition. In some cases, the endophytes, parts, or propagules thereof can be used concomitantly with one or more of the other agrichemicals such as various pesticides, acaricides, nematicides, other types of fungicides, and plant growth regulators.

Various types of additional fungicides can optionally be included in the compositions described herein. Examples include copper fungicide such as basic copper chloride and basic copper sulfate, sulfur fungicide such as thiuram, zineb, maneb, mancozeb, ziram, propineb, and polycarbamate, polyhaloalkylthio fungicide such as captan, folpet, dichlorfluanid, organochlorine fungicide such as chlorothalonil, fthalide, organophosphorous fungicide such as O,O-bis(1-methylethyl) S-phenylmethyl phosphorothioate (IBP), edifenphos (EDDP), tolclophos-methyl, pyrazophos, fosetyl, dicarboxylmide fungicide such as iprodione, procymidone, vinclozolin, fluoromide, carboxyamide fungicide such as oxycarboxin, mepronil, flutolanil, tecloftalam, trichlamide, pencycuron, acylalanine fungicide such as metalaxyl, oxadixyl, furalaxyl, methoxyacrylate fungicides such as kresoxim-methyl (stroby), azoxystrobin, metominostrobin, trifloxystrobin, pyraclostrobin, anilinopyrimidine fungicide such as andupurine, mepanipyrim, pyrimethanil, cyprodinil, antibiotic agents such as polyoxin, blasticidin S, kasugamycin, validamycine, dihydrostreptomycin sulfate, propamocarb hydrochloride, quintozene, hydroxyisoxazole, methasulfocarb, anilazine, isoprothiolane, probenazole, chinomethionat, dithianon, dinocap, diclomezine, ferimzone, fluazinam, pyroquilon, tricyclazole, oxolinic acid, iminoctadine acetate, iminoctadine albesilate, cymoxanil, pyrrolnitrin, diethofencarb, binapacryl, lecithin, sodium bicarbonate, fenaminosulf, dodine, dimethomorph, phenazine oxide, carpropamide, flusulfamide, fludioxonil, famoxadone, or combinations thereof. Hence, other types of fungicides can be mixed together with and used in various amounts with one or more of the extracts or compounds described herein.

The endophytes, parts, or propagules thereof described herein can be used in a weight ratio relative to the other type of fungicide such as from 1:0.001 to 1:1000 as a weight ratio. In some instance, the amount of endophytes, parts, or propagules thereof relative to the other type of fungicide can vary from 1:0.01 to 1:100 as a weight ratio within a composition.

Pesticides can be included in the compositions, with any of the endophytes, parts, or propagules described herein. The pesticides can include organophosphorous pesticides, carbamate pesticides such as fenthion, fenitrothion, diazinon, chlorpyrifos, ESP, vamidothion, phenthoate, dimethoate, formothion, malathon, trichlorfon, thiometon, phosmet, dichlorvos, acephate, EPBP, methylparathion, oxydemeton-methyl, ethion, salithion, cyanophos, isoxathion, pyridaphenthion, phosalone, methidathion, sulprofos, chlorfevinphos, tetrachlorvinphos, dimethylvinphos, propaphos, isofenphos, ethylthiometon, profenofos, pyraclofos, monocrotophos, azinphosmethyl, aldicarb, methomyl, thiodicarb, carbofuran, carbosulfan, benfuracarb, furathiocarb, propoxur, BPMC, MTMC, MIPC, carbaryl, pirimicarb, ethiofencarb, and fenoxycarb, pyrethroid pesticides such as permethrin, cypermethrin, deltamethrin, fenvalerate, fenpropathrin, pyrethrin, allethrin, tetramethrin, resmethrin, dimethrin, propathrin, phenothrin, prothrin, fluvalinate, cyfluthrin, cyhalothrin, flucythrinate, ethofenprox, cycloprothrin, tralomethrin, silafluofen, brofenprox, and acrinathrin, and benzoylurea and other types of pesticides such as diflubenzuron, chlorfluazuron, hexaflumuron, triflumuron, tetrabenzuron, flufenoxuron, flucycloxuron, buprofezin, pyriproxyfen, methoprene, benzoepin, diafenthiuron, acetamiprid, imidacloprid, nitenpyram, fipronil, cartap, thiocyclam, bensultap, nicotin sulfate, rotenone, mataldehyde, machine oil, and microbial pesticides e.g., BT and insect pathogenic virus.

Acaricides can be included in the compositions described herein. The acaricides that can be employed include, for example, chlorbenzilate, phenisobromolate, dicofol, amitraz, BPPS, benzomate, hexythiazox, fenbutatin oxide, polynactin, chinomethionat, CPCBS, tetradifon, avermectin, milbemectin, clofentezin, cyhexatin, pyridaben, fenpyroximate, tebufenpyrad, pylidimifen, fenothiocarb, and dienochlor.

As for the aforementioned nematicides, fenamiphos, fosthiazate and the like can be specifically exemplified; as for plant-growth regulators, gibberellins (e.g., gibberellin A3, gibberellin A4, and gibberellin A7), auxin, 1-naphthaleneacetic acid, and so on can be specifically exemplified.

More generally, the endophyte, parts, or propagules thereof can be combined with any solid or liquid additive, which complies with acceptable formulation techniques. In general, the composition according to the invention may contain from 0.05 to 99% by weight of endophytes, parts, or propagules thereof, preferably from 10 to 70% by weight.

The endophytes, parts or propagules thereof, or compositions thereof can be provided in a ready-to-use form that can be prepared for use. The endophytes, parts, or propagules thereof, or compositions thereof can be applied by a suitable device, such by use of a spraying or dusting device. The endophytes, parts, or propagules thereof, or compositions thereof can be applied by use of brush or roller.

The endophytes, parts, or propagules or compositions thereof can be provided in concentrated commercial compositions that should be diluted before application to the crop. For example, the endophytes, parts, or propagules thereof, or compositions thereof can be provided in dry (e.g., lyophilized) form, or in concentrated form, and then dissolved or diluted as desired. The endophytes, parts, or propagules thereof, or compositions thereof can be formulated as an aerosol, as a cold fogging concentrate, as a dustable powder, as an emulsifiable concentrate, as an oil in water emulsion, as a water in oil emulsion, as an encapsulated granule, as a fine granule, as a flowable concentrate for seed or nut treatment, as a gas (under pressure), as a gas generating product, as granules, as a hot fogging concentrate, as macrogranules, as microgranules, as an oil dispersible powder, as an oil miscible flowable concentrate, as an oil miscible liquid, as a paste, as a plant rodlet, as a powder for dry seed or nut treatment, as seeds or nuts coated with the composition, as a soluble concentrate, as a soluble powder, as a solution for seed (or other) treatment, as a suspension concentrate (flowable concentrate), as an ultra-low volume (ULV) liquid, as an ultra-low volume (ULV) suspension, as water dispersible granules, as a water dispersible powder for slurry treatment, as water soluble granules or tablets, as a water soluble powder for seed or nut treatment, as a wettable powder, or as a combination thereof (e.g., two types of formulations packaged together).

For example, the compositions can include a carrier and the conidia, spores, propagules, or a combination thereof, from at least two of the following endophytes: Alternaria destruens, Fusarium commune, Fusarium oxysporum, or a combination thereof. In some cases, the conidia, spores, propagules, or a combination thereof can be dried and/or encapsulated. For example, the carrier can include corn starch, rice flour, talc, diatomaceous earth, kaolin, plant oil, or a combination thereof.

The following Examples illustrate some of the experimental work involved in the development of the invention.

### EXAMPLE 1

### Materials and Methods

This Example illustrates some of the materials and methods employed in the development of the invention

### Fungal Strains and Growth Conditions; Wheat and Millet Cultivars

Fusarium and Alternaria endophytes were isolated from wheat stems or heads in 2013 and identified to the genus level by sequencing the internal transcribed spacer region (Gdanetz and Trail 2017).

F. graminearum isolates used in in vitro competition experiments were collected. F. graminearum isolate PH-1 (FGSC 9075; NRRL 31084) (Trail and Common 2000) was the first F. graminearum strain to have its genome sequenced (Cuomo et al. 2007) and has been used worldwide to study head blight disease. F. graminearum isolates used in in vitro competition experiments were collected as indicated in Table 1.

TABLE 1
Fusarium graminearum isolates

| Isolate ID | Year Collected | Origin | Host | County |
|---|---|---|---|---|
| 107M | 2017 | wheat head | wheat | Presque Isle |
| 107H | 2017 | wheat head | wheat | Presque Isle |
| 24A | 2016 | wheat head | wheat | Huron |
| 76J | 2017 | wheat head | wheat | Lenawee |
| 760 | 2017 | wheat head | wheat | Lenawee |
| 93D | 2017 | wheat head | wheat | Sanilac |
| PH-1 | 1995 | corn stalk | corn | Ingham |

F. graminearum isolate PH-1 can produce DON and 15-ADON forms of deoxynivalenol (Alexander et al. 2011). The other six isolates were chosen from a survey of Michigan to represent a diversity of locations. These isolates were confirmed to be F. graminearum by sequencing of translation elongation factor 1-α (TEF1-α). Unless otherwise noted, all isolates were stored at −80° C. as colonized malt extract agar (MEA) blocks in 35% glycerol and were commonly grown on 2% MEA medium.

The wheat cultivar Wheaton is a susceptible spring wheat variety that was used throughout the study. Proso millet (Panicum miliaceum) was used for generating inoculum for plant inoculation (Gdanetz and Trail 2017).

### Molecular Identification of Endophytes

Endophytes were grown on MEA for five days. Three plugs of the endophytes were then transferred to Erlenmeyer flasks containing 100 ml yeast extract peptone dextrose (YEPD) broth. Mycelium was harvested after three days of growth in YEPD broth and 50 mg mycelia were ground with 0.08 ml of lysing matrix A (MP Biomedicals, Houston, TX) and a four-millimeter diameter steel ball (SPEX SamplePrep, Metuchen, NJ) using a FastPrep FP120 (Thermo Fisher Scientific, Waltham, MA) to prepare endophyte lysates. Genomic DNA (gDNA) was extracted from the endophyte lysates using the Dneasy plant Mini Kit (Qiagen Sciences Inc., Germantown, MA, USA) following the manufacturers' instructions.

Two endophytes in the Fusarium genus were identified to species level by amplifying and sequencing the translation elongation factor 1-α (TEF1-α), along with the two subunits of the RNA polymerase II gene (RPB1 and RPB2). TEF1-α was amplified using the primers EF1 and EF2. RPB 1 was amplified and sequenced in two overlapping segments using the primer pairs Fa with R8 and F7 with R9 (O'Donnell et al. 2010; Hofstetter et al. 2007). The first half of RPB2 (RPB2-1) was amplified and sequenced using the primer pair 5f2 and 7cr (Reeb et al. 2004) (Liu et al. 1999). The second half of RPB2 (RPB2-2) was amplified and sequenced using the primer pair 7cf and 11ar. The endophyte identified within the Alternaria genus was identified by amplifying the plasma membrane ATPase and calmodulin genes, as suggested by Lawrence et al. (Lawrence et al. 2013). Primers for the ATPase were the ATPDF1 and ATPDR1. The calmodulin gene was amplified using the primer pairs CALDF1 and CALDR1. Primer sequences are shown in Table 2.

TABLE 2
Primers

| Primer | | |
|---|---|---|
| name | Locus Targeted | Sequence (5′-3′) |
| TEF1 | translation | ATGGGTAAGGARGACAAGAC |
| | elongation | (SEQ ID NO: 9) |
| | factor 1-α | |
| TEF2 | translation | GGARGTACCAGTSATCATG |
| | elongation | (SEQ ID NO: 10) |
| | factor 1-α | |
| Fa | RPB1 | CAYAARGARTCYATGATGGGWC |
| | | (SEQ ID NO: 11) |
| R8 | RPB1 | CAATGAGACCTTCTCGACCAGC |
| | | (SEQ ID NO: 12) |
| F7 | RPB1 | CRACACAGAAGAGTTTGAAGG |
| | | (SEQ ID NO: 13) |
| R9 | RPB1 | TCARGCCCATGCGAGAGTTGTC |
| | | (SEQ ID NO: 14) |
| 5f2 | RPB2 | GGGGWGAYCAGAAGAAGGC |
| | | (SEQ ID NO: 15) |
| 7cr | RPB2 | CCCATRGCTTGYTTRCCCAT |
| | | (SEQ ID NO: 16) |
| 7cf | RPB2 | ATGGGYAARCAAGCYATGGG |
| | | (SEQ ID NO: 17) |
| 11ar | RPB2 | GCRTGGATCTTRTCRTCSACC |
| | | (SEQ ID NO: 18) |
| ATPDF1 | ATPase | ATCGTCTCCATGACCGAGTTCG |
| | | (SEQ ID NO: 19) |
| ATPDR1 | ATPase | TCCGATGGAGTTCATGATAGCC |
| | | (SEQ ID NO: 20) |
| CALDF1 | Calmodulin | AGCAAGTCTCCGAGTTCAAGG |
| | | (SEQ ID NO: 21) |
| CALDR1 | Calmodulin | CTTCTGCATCATCAYCTGGACG |
| | | (SEQ ID NO: 22) |

Polymerase chain reaction (PCR) was performed using a final concentration of 1× Phusion Green HotStart II High-Fidelity DNA Polymerase (Thermo Scientific) containing 0.5 μM forward and reverse primers, for each respective locus, and 10-40 ng genomic DNA. Thermal cycling conditions for EF1-α, RPB2-1, and RPB2-2 were as follows: 98° C. for 30 sec, followed by 35 cycles of 98° C. for 10 sec, 59° C. for 30 seconds, and 72° C. for 1.5 min, followed by a final extension at 72° C. for 7 min. RPB1 was amplified using the same thermal cycling conditions except for the annealing temperature at 57° C. (Fa and R8) or 61° C. (F7 and R9). Amplicons were separated by gel electrophoresis and successfully amplified amplicons were purified via gel extraction with the Wizard® SV Gel and PCR Clean-up System (Promega, Madison, WI) or by adding 3 U exonuclease I and 0.5 U shrimp alkaline phosphatase (Thermo Scientific, Waltham, MA) and incubating at 37° C. for 45 min followed by 85° C. for 10 min to inactivate enzymes. Amplicons were Sanger-sequenced at the Michigan State University Genomics core facility (website rtsf.natsci.msu.edu/genomics/). Forward and reverse sequences were trimmed and assembled with Codon Code Aligner v4.2.7 and consensus sequences were compared against a curated set of Fusarium species using the CBS-KNAW Fungal Biodiversity Centre's Fusarium Multilocus Sequence Typing database (website knaw.nl/fusarium/).

Fusarium RPB1 and RPB2, and Alternaria ATPase and calmodulin gene sequences were used for phylogenetic analyses. RPB1 and RPB2 sequences of Fusarium endophytes were aligned to sequences from Fusarium species within the Gibberella clade (O'Donnell et al. 2013) using MUSCLE v3.8.31. Alternaria ATPase and calmodulin gene sequences were aligned to Alternaria species within sections Alternaria, Sonchi, and Alternantherae (Lawrence et al. 2013) using MUSCLE v3.8.31. The Markov Chain Monte Carlo (MCMC) algorithm was implemented in MrBayes v3.2.6 (Ronquist et al. 2012) to generate a Bayesian phylogeny using the combined RPB1 and RPB2 or ATPase and calmodulin alignments treating each gene sequence as a separate partition. The MCMC algorithm was run with a GTR+I+G molecular model of evolution, 5,000,000 generations, trees sampled every 1000 generations, and a 25% burn-in.

### Competition of Endophytes in vitro

Colonies of the six F. graminearum isolates and endophytes were cultured separately on MEA in constant light. After five days, a 5-mm plug of each was placed mycelial side down three centimeters apart on MEA agar medium within 100-mm Petri dishes. Dual cultures were incubated in the dark at 25° C. for 18 days. Photographs were taken of each Petri dish at 4, 11, 14, and 18 days post-inoculation. A 1-cm⁻² calibration card was included within each image to convert pixels to distances within the image analyzer Fiji (Schindelin et al. 2012). The area occupied by F. graminearum was estimated from the images by tracing the colony area. Petri dishes containing only F. graminearum (no endophyte) served as a negative control.

To test for the effect of potential endophyte volatiles on the growth of F. graminearum, the pathogen and endophytes were grown physically separated on MEA medium, as described above, and a 5-mm plug of each was placed mycelium side down, on separate 60 mm Petri dishes containing MEA medium. Two Petri dishes containing agar medium with endophytes and one Petri dish containing agar medium with F. graminearum were placed within an empty 150 mm Petri dish. Fungi were incubated within the larger Petri dishes in the dark at 25° C. for six days. Images were taken six days post-inoculation and the colony area was estimated as described above.

### Preparation of Inoculum for Soil Infestation

The inoculum was prepared by growing F. grarninearum PH-1 in spawn bags containing moistened seeds of proso millet. Briefly, white millet seeds (1 kg) were covered with sterile water and allowed to imbibe for 24 h at room temperature, distributed into 2 spawn bags (ECAB2430; Fungi perfecti, Olympia WA), and sterilized for 45 min at 121° C. in an autoclave. Millet was allowed to cool and stored at 4° C. until use. F. graminearum conidia were spread onto Petri dishes (100 mm×15 mm) containing potato dextrose agar (PDA) with chloramphenicol (0.17 mg ml⁻¹), and erythromycin (0.25 mg ml⁻¹), and grown for 7 days at room temperature in constant fluorescent light. One Petri dish colonized with F. graminearum PH-1 culture was combined with one plate of fresh PDA and 130 ml dH₂O then homogenized in a sterilized glass blender. The slurry (120ml) was then poured into a spawn bag containing the sterilized millet. The bag was sealed, the contents mixed, and left under continuous fluorescent light at room temperature for 10 days. Following incubation, the contents of the bag were air-dried in a flow cabinet at room temperature for 3 days and then maintained at 4° C. until use.

### Pretreatment of Wheat Seeds With Endophytes

Agar plugs colonized with a single endophyte were placed in the center of MEA-containing Petri dishes (60 mm×15 mm) and grown under continuous fluorescent light for four to five days. Seeds were surface-sterilized first by soaking in 95% ethanol for 10 sec, rinsing with sterile water three times, soaking in 0.4% sodium hypochlorite containing 0.01% Tween-20 for three min, and finally rinsing three times with sterile water. Surface sterilized wheat seeds were placed on the edges of the colonies and incubated for three days; control seeds were placed on MEA which did not contain an endophyte. Germinated seeds were planted one seed per cone-tainer (50 ml; Steuwe and Sons, Inc., Tangent, OR) containing an equal mix of non-autoclaved potting soil (Suremix Perlite, Michigan GrowerProducts, Inc., Galesburg, MI) and field soil (agricultural field in Mason MI) with or without 5% F. graminearum PH-1 inoculum.

### Treatment of Wheat Heads With Endophytes and F. graminearum

Wheat plants, cultivar Wheaten (four per 9″ pot) were grown in the greenhouse at 21-22° C. with supplemental lighting and inoculated as previously described (Guenther and Trail 2005). Conidia were produced by growing each endophyte on MEA for one week then rubbing off the conidia with a bent glass rod, rinsing the rod with 0.05% Tween 20 in water, collecting the solution of conidia/spores, and centrifuging the collected solution to separate the spores. The supernatant was removed, and spores were suspended in 35% glycerol to 2.8×10⁵ conidia ml⁻¹.

Approximately four to five weeks after planting, wheat heads were at the beginning of anthesis (defined as 50% heads producing visible anthers) and were pre-treated with endophytes by pipetting 30 μl fresh conidia on the rachis, between florets, and on awns. The heads were covered with a glassine pollination/grain bag (Canvasback® G27, Seedburo Company, Des Plaines IL) to keep humidity high. After six days the heads were infected with F. graminearum PH-1 by pipetting 10 μl conidia (1.5×10⁵ conidia ml⁻¹ in 35% glycerol) between the lemma and palea of a single floret central per head. Wheat heads were similarly inoculated with 35% glycerol without fungi to serve as negative controls, and heads inoculated with F. graminearum PH-1 served as a positive control. Watering of plants was stopped after four weeks, when the seeds were filled, to allow the seeds to dry. After three additional weeks, the mature seeds were harvested from each head, air-dried, and weighed. Seed weight (i.e., average seed weight per head) was analyzed with a linear mixed model with the treatment as a fixed effect and biological replicate as a random factor. Additionally, seeds were analyzed for mycotoxins.

### Mycotoxin Quantification in Wheat Seeds

Determination of DON and 15-ADON concentration in seeds from greenhouse trials was conducted by Dr. Yanhong Dong at the Mycotoxin Diagnostic Laboratory and extracted in the Department of Plant Pathology, University of Minnesota, St. Paul, using gas chromatography-mass spectrometry (GC-MS) as described by Mirocha et al. (1998). Mycotoxin data were analyzed with a Kruskal-Wallis test followed by pairwise comparisons using the Wilcoxon rank-sum test with a Benjamini-Hochberg p-value adjustment to control the false discovery rate.

### Statistical Analysis and Data Availability

All statistical analysis was conducted in Rv3.5.2 using the packages ‘lme4’ (Bates et al. 2015) and ‘emmeans’ (Lenth 2016). All data and R code used in this manuscript are available on GitHub (website at github.com/noelzach/EndophyteBiocontrol) or upon request. TEF1-α, RPB1, RPB2, ATPase, and calmodulin gene sequences were deposited in GenBank under the accession numbers MW917147-MW917154.

### EXAMPLE 2

### Molecular Identification of Endophytes

Three endophytes isolated from wheat microbiomes and shown to have antagonistic effects towards F. graminearum were phylogenetically characterized. The ATPase gene (1194 bp) from Alternaria endophyte #37 had a 99.83% match with 100% coverage to Alternaria destruens CBS 121454. This ATPase gene from Alternaria endophyte #37 had the following sequence (SEQ ID NO:1).

```
      1 TACATCCTCT TCTACCGCGA CACCCGAACC AACCCTCACG
    41 CCGAGCAAAC CACCAAGAAG AAGGCCTGGT GGCAGTTCTG
    81 GAAGTCTGGC TCAGCTACCG CTGCCACTCC CATCCAGGAT
  121 GCCGGTGCCG TCCCCGACGA CTGTAAGTTT TATCATCCTG
  161 CTCACTCGAT TGCATGCACC TGCATCACAT AGCACTGCTG
  201 TTTGCGGCAG CGCTCAACGT ACCTCGCCAA TTCATCCTTT
  241 GTTGAGCTTT ACCTCGACAT TTGGTGGCTG GCATGGTCCG
  281 CGCTCAAGCT GCTCCCTGCT AGCGACGCGA TAGCGGCAGA
  321 AATGGTGGAG CCAATCATGC AATCCGGCTC CACCAAACTA
  361 CCCGCTTCTG CAGCATCCGA AATGAGCAAC ACGATCAAGA
  401 GGAATTTTGC TAACATGGAA TTGCAGACCT CAACACTGAG
  441 CTCCGAACTG GTCTCACCTC GTCCGACGTT GAGCAGCGTC
  481 GCAAGCGCTA TGGTTTCAAC GAAATCTCTT CTGAGAAGAC
  521 CAACCTTCTC AAGCAGTTCA TCGGTTACTT CACTGGTCCC
  561 ATTCTCTACG GTAAGCATCC CTGCACAAAC TTGTTTAGCG
  601 CCAAACTAAC GCATCATAGT CATGGAGCTC GCTGCTCTTC
  641 TCGCCGCTGG TCTTCAGGAT TGGGTCGATT TCGGTGTCAT
  681 CTGCGGTATC CTGTTGCTCA ACGCCATCGT CGGTTGGTAC
  721 CAGGAGAAAC AGGCTGCTGA TGTCGTCGCT TCGCTCAAGG
  761 GTGATATCGC CATGAAGGCC ACCGTCGTTC GTGACAACCA
  801 GCAACAGACC ATTCTCGCTC GTGAGCTTGT TCCCGGTGAC
  841 ATCGTCGTTA TTGAGGAGGG TCAATCCGTC CCCGGTGACG
  881 CCCGTCTTAT CTGCGGCTAC GACCACCCTG AGGACTTCGA
  921 CTTGTACATG AAGCTCAAGG CTGAGGACAA GTTCCACGAC
  961 GCTGACCCCG AGGACGAGAA GGATGACGAC GTCGATGAGG
1001 AGAAGTTCGA CGAGGAGAAC CCCATCACTC AGGGCCACCC
1041 TCTCGTTGCT TGCGATCAAT CGTCCATCAC CGGAGAGTCT
1081 CTCGCTGTCG ACAAGTACAT GGGAGAAGTC GCCTACTACA
1121 CCACTGGTTG CAAGCGCGGC AAGGCCTACG GTATCGTCAT
1161 CACCACTGCT AAGCACTCTT TCGTCGGTCG CACT
```

The Alternaria destruens CBS 121454 ATPase has the following protein sequence (NCBI QC069008.1; SEQ ID NO:23).

```
    1 YILFYRDTRT NPHAEQTTKK KAWWQFWKSG SATAATPIQD
  41 AGAVPDDYLN TELRTGLTSS DVEQRRKRYG FNEISSEKTN
  81 LLKQFIGYFT GPILYVMELA ALLAAGLQDW VDFGVICGIL
121 LLNAIVGWYQ EKQAADVVAS LKGDIAMKAT VVRDNQQQTI
161 LARELVPGDI VVIEEGQSVP GDARLICGYD HPEDFDLYMK
201 LKAEDKFHDA DPEDEKDDDV DEEKFDEENP ITQGHPLVAC
241 DQSSITGESL AVDKYMGEVA YYTTGCKRGK AYGIVITTAK
281 HSFVGRT
```

A nucleotide sequence for the Alternaria destruens CBS 121454 ATPase is shown below (NCBI MH092839.1; SEQ ID NO:24).

```
      1 TACATCCTCT TCTACCGCGA CACCCGAACC AACCCTCACG
    41 CCGAGCAAAC CACCAAGAAG AAGGCCTGGT GGCAGTTCTG
    81 GAAGTCTGGC TCAGCTACCG CTGCCACTCC CATCCAGGAT
  121 GCCGGTGCCG TCCCCGACGA CTGTAAGTTT TACCATCCTG
  161 CTCACTCGAT TGCATGCACC TGCATCACAT AGCACTGCTG
  201 TTTGCGGCAG CGCTCAACGT ACCTCGCCAA TTCATCCTTT
  241 GTTGAGCTTT ACCTCGACAT TTGGTGGCTG GCATGGTCCG
  281 CGCTCAAGCT GCTCCCTGCT AGCGACGCGA TAGCGGCAGA
  321 AATGGTGGAG CCAATCATGC AATCCGGCTC CACCAAACTA
  361 CCCGCTTCTG CAGCATCCGA AATGAGCAAC ACGATCAAGA
  401 GGCATTTTGC TAACATGGAA TTGCAGACCT CAACACTGAG
  441 CTCCGAACTG GTCTCACCTC GTCCGACGTT GAGCAGCGTC
  481 GCAAGCGCTA TGGTTTCAAC GAAATCTCTT CTGAGAAGAC
  521 CAACCTTCTC AAGCAGTTCA TCGGTTACTT CACTGGTCCC
  561 ATTCTCTACG GTAAGCATCC CTGCACAAAC TTGTTTAGCG
  601 CCAAACTAAC GCATCATAGT CATGGAGCTC GCTGCTCTTC
  641 TCGCCGCTGG TCTTCAGGAT TGGGTCGATT TCGGTGTCAT
  681 CTGCGGTATC CTGTTGCTCA ACGCCATCGT CGGTTGGTAC
  721 CAGGAGAAAC AGGCTGCTGA TGTCGTCGCT TCGCTCAAGG
  761 GTGATATCGC CATGAAGGCC ACCGTCGTTC GTGACAACCA
  801 GCAACAGACC ATTCTCGCTC GTGAGCTTGT TCCCGGTGAC
  841 ATCGTCGTTA TTGAGGAGGG TCAATCCGTC CCCGGTGACG
  881 CCCGTCTTAT CTGCGGCTAC GACCACCCTG AGGACTTCGA
  921 CTTGTACATG AAGCTCAAGG CTGAGGACAA GTTCCACGAC
  961 GCTGACCCCG AGGACGAGAA GGATGACGAC GTCGATGAGG
1001 AGAAGTTCGA CGAGGAGAAC CCCATCACTC AGGGCCACCC
1041 TCTCGTTGCT TGCGATCAAT CGTCCATCAC CGGAGAGTCT
1081 CTCGCTGTCG ACAAGTACAT GGGAGAAGTC GCCTACTACA
1121 CCACTGGTTG CAAGCGCGGC AAGGCCTACG GTATCGTCAT
1161 CACCACTGCT AAGCACTCTT TCGTCGGTCG CACT
```

The Alternaria endophyte #37 also had a calmodulin gene (776 bp) with a one base pair mismatch and 100% coverage compared to Alternaria destruens CBS 121454 and Alternaria lini CBS 106.34. This calmodulin gene from Alternaria endophyte #37 had the following sequence (SEQ ID NO:2).

```
    1 CGTAAGTGCC CTCCCCATCC TCTGCCATGC CGCGCGGCTG
  41 CCTGGTAGCC CTGGGGGCCT GCGCAATCAC GAACATGCAG
  81 CTGACGACGT CGTGTTGTAG GACAAGGATG GCGATGGTCA
121 GTACTCTCCC TCCAAATTCC CTTCCACACA CACACTCTCT
161 CTCCCTCTCT GCCTTCAAAG CAGTGCCGCA TCTCCAGCCT
201 ACGCAATCGG CAGAGGGGCC CGGGCGAGGC TTGCTGGCTA
241 GGGGTCCAAA CCACCGCCCA CAGCTACAAC ACCACGACAT
281 CCACCCTACT CCATAGCAAG CACAACTGAC GACGATGCGC
321 CACAGGTCAA ATCACCACCA AGGAGCTAGG TACCGTCATG
361 CGCTCGCTCG GCCAAAATCC CAGCGAGTCT GAGCTCCAGG
401 ACATGATCAA CGAGGTCGAT GCCGACAACA ACGGCACCAT
441 TGACTTCCCA GGTGCGCCCC TTCATACCAG TCCAAAGTAC
481 CACAGCTAAC TTTTCCAGAA TTCCTTACCA TGATGGCCCG
521 CAAGATGAAG GACACCGACT CCGAGGAGGA GATCCGGGAA
561 GCCTTCAAGG TCTTCGACCG CGATAACAAC GGTTTCATCT
601 CCGCCGCCGA ACTGCGTCAC GTCATGACTT CTATTGGCGA
641 GAAATTGACC GATGACGAGG TCGACGAGAT GATCCGGGAG
681 GCTGACCAGG ACGGTGACGG CCGCATCGAC TGTAGGTTAC
721 AGCTGCCTAT ATCACGAGTG CGATGCTAAC ACACATCAGA
761 CAACGAGTTC GTCCA
```

The Alternaria destruens CBS 121454 calmodulin nucleotide sequence is shown below as SEQ ID NO:25 (NCBI MH175186.1).

```
    1 CGTAAGTGCC CTCCCCATCC TCTGCCATGC CGCGCGGCTG
  41 CCTGGTAGCC CTGGGGGCCT GCGCAATCAC GAACATGCAG
  81 CTGACGACGT CGTGTTGTAG GACAAGGATG GCGATGGTCA
121 GTACTCTCCC TCCAAATTCC CTTCCACACA CACACTCTCT
161 CTCCCTCTCT GCCTTCAAAG CAGTGCCGCA TCTCCAGCCT
201 ACGCAATCGG CAGAGGGGCC CGGGCGAGGC TTGCTGGCTA
241 GGGGTCCAAA CCACCGCCCA CAGCTACAAC ACCACGACAT
281 CCACCCTACT CCATAGCAAG CACAACTGAC GACGATGCGC
321 CACAGGTCAA ATCACCACCA AGGAGCTAGG TACCGTCATG
361 CGCTCGCTCG GCCAAAATCC CAGCGAGTCT GAGCTCCAGG
401 ACATGATCAA CGAGGTCGAT GCCGACAACA ACGGCACCAT
441 TGACTTCCCA GGTGCGCCCC TTCATACCAG TCCAAAGTAC
481 CACAGCTAAC TTTTCCAGAA TTCCTTACCA TGATGGCCCG
521 CAAGATGAAG GACACCGACT CCGAGGAGGA GATCCGGGAA
561 GCCTTCAAGG TCTTCGACCG CGATAACAAC GGTTTCATCT
601 CCGCCGCCGA ACTGCGTCAC GTCATGACTT CTATTGGCGA
641 GAAATTGACC GATGACGAGG TCGACGAGAT GATCCGGGAG
681 GCTGACCAGG ACGGTGACGG CCGCATCGAC TGTAGGTTAC
721 AGCTGCCTAT ATCACAAGTG CGATGCTAAT ACACACCAGA
761 CAACGAG
```

The Alternaria destruens CBS 121454 calmodulin encodes a protein with the following sequence (SEQ ID NO:26).

```
    1 DKDGDGQITT KELGTVMRSL GQNPSESELQ DMINEVDADN
  41 NGTIDFPEFL TMMARKMKDT DSEEEIREAF KVFDRDNNGF
  81 ISAAELRHVM TSIGEKLTDD EVDEMIREAD QDGDGRIDYN
121 E
```

According to Woundenberg et al. (Woudenberg et al. 2015), both of Alternaria destruens CBS 121454 and Alternaria lini CBS 106.34 are synonyms for Alternaria alternata. In the application, Alternaria endophyte #37 is referred to as Alternaria destruens. This name is corroborated with the phylogenetic placement since Alternaria endophyte #37 is grouped in a well-supported (posterior probability 1.0) clade with Alternaria destruens CBS 121454 (FIG. 1A). Bayesian phylogeny of Alternaria ATPase and calmodulin genes with Alternaria endophyte #37 are shown in FIG. 1A.

Fusarium endophyte #40 had an RPB1 sequence exactly matched (100% identical, 100% coverage) the RPB1 from Fusarium commune NRRL 28387. The RPB2 sequence for #40 had 99.76% sequence homology with 92.73% overlap with the RBP2 from two Fusarium commune isolates (NRRL 13816 and NRRL 28058). Additionally, the EF1-α sequence had an exact match (100% identical, 100% overlap) to the EF1-α sequence of two Fusarium commune isolates (NRRL 28058 and NRRL 13816). The identification of Fusarium endophyte #40 as being Fusarium commune was corroborated by phylogenetic placement because it grouped in a well-supported (posterior probability=1.0) with Fusarium commune NRRL 28387 (FIG. 1B).

The Fusarium endophyte #40 isolate had the following RPB1 gene sequence (SEQ ID NO:3), which had 100% sequence identity (with 100% coverage) compared to a RPB1 from Fusarium commune NRRL 28387.

```
      1 TTTTCCTCAC AAAGGAGCAA ATCATGAACT GTATGCTCTG
    41 GGTGCCTAAC TGGGACGGTG TCATTCCTCA ACCCGCTATC
    81 TATAAGCCTC GTCCTCGGTG GACTGGTAAG CAGCTTATCA
  121 GCATGGTTAT CCCTAAGGAG GTTAGCCTGT TCAACGGTAC
  161 GGATTCTGGT GAAAACGCCC CTCTTAAGGA CGAGGGTCTT
  201 CTGATCCAAG CTGGCCAACT GATGTATGGT CTTTTGACTA
  241 AGAAGAACAT TGGTGCTGCT GCGGGCGGTA TTGTGCATAT
  281 CAGCTACAAC GAACTTGGCC CCGAAGGTGC GATGGCTTTC
  321 TTGAACGGTG TCCAGCAGGT TGTCACCTAC TGGCTTCTCA
  361 ACAATGGCCA TAGCATTGGT ATTGGTGATA CAATTCCCGA
  401 TGCGGCGACC ATTGCTAAAG TTCAGGTACA TATTGATGAG
  441 GAAAAGGCTG AAGTTGCTCG CTTGACAGCA ATGGCCACAG
  481 CGAATGAGCT TGAGGCCCTA CCTGGTATGA ACGTTCGTGC
  521 AACCTTCGAA AACAAGGTCT CCATGGCTCT GAACCAGGCC
  561 CGTGATAAGG CTGGTACCAC AACACAGAAG AGTTTGAAGG
  601 ATTCAAACAA CGCTGTCACC ATGGCTTCCT CAGGTTCCAA
  641 GGGTTCATCT ATCAATATTT CTCAAATGAC TGCGCTTGTC
  681 GGTCAGCAAA TTGTCGAAGG CAAGCGTATT CCTTTTGGTT
  721 TCAAGTATCG CACATTACCT CACTTCACCA AGGACGATTA
  761 CTCACCTGAG GCCCGTGGCT TCGTCGAGAA CTCTTACCTC
  801 CGTGGTCTCA CTCCCAGCGA GTTTTTCTTC CACGCCATGG
  841 CTGGTCGAGA AGGTCTCATT GATACTGCAG TCAAGACTGC
  881 CGAAACAGGT TATATCCAGC GACGATTGGT CAAGGCTCTG
  921 GAAGATCTTT CTGCCCGTTA CGATGGAACT GTCCGAAACT
  961 CTCTGGGAGA CATTGTTCAG TTCCTCTATG GTGAAGACGG
1001 TCTCGATGCC ATGATTATTG AGAAACAGAA GTTGGGTATC
1041 CTCAATATGT CAAACTCGGC ATTTGAAAAG AAGTATCGTC
1081 TGGATCTTGC CAACCCCCCG GACTGGTTTA AGCACGACTA
1121 CGAATTCGGT AATGAATTGA CTGGTGACAA GGAATCTATG
1161 GAGTATCTCG ATCAAGAATG GGAAAAGTTG TTGGCTGATC
1201 GCAGACAAAT CCGACAGATC AACAAGGCCA AGGGTAACGA
1241 GGAAATGATG CAACTGCCCC TCAACATCAC TCGCATCATC
1281 GAGTCTGCTA AGCGAGTCTT TAATGTCAAG GCTAATGACC
1321 GAAGCAACTT GCGACCTTCG GAAGTTATTC CAGCTGTGCA
1361 AAACTTGTTG GATAGCATGA AGATTGTTCG TGGTACTGAT
1401 GAAATCTCGA TTGAAGCTGA CGCAAATGCA TCCATTCTCT
1441 TCAAGGCCTT GCTTCGCTCT CGCCTGGCCT TCAAGGAGGT
1481 GGTCAAGGAG CACCGGTTGA ACAAATTAGC TTTCGACCAT
1521 ATTCTGGGTG AACTCCAGAA TAGATGGGAT CGCGCATTTG
1601 TCA
```

The Fusarium endophyte #40 isolate had the following RPB2 gene sequence (SEQ ID NO:4), which had 99.76% sequence identity to a RPB2 from two Fusarium commune isolates (NRRL 13816 and NRRL 28058).

```
      1 TTGACAGATA TACCTTTGCT TCGACTCTTT CACATTTGCG
    41 TCGAACCAAT ACTCCTATTG GACGAGATGG TAAATTGGCC
    81 AAGCCTCGAC AGCTTCACAA TACTCACTGG GGTTTGGTGT
  121 GTCCCGCCGA AACGCCTGAG GGTCAAGCTT GTGGTCTGGT
  161 CAAAAACTTG TCTCTGATGT GTTATGTCAG TGTCGGCTCT
  201 CCAGCCGAGC CTCTCATTGA ATTCATGATC AACAGAGGTA
  241 TGGAAGTTGT TGAGGAGTAC GAGCCGACAA GATATCCCCA
  281 CGCTACAAAG ATTTTCGTCA ACGGTAGCTG GGTTGGTGTT
  321 CACGCCGACC CCAAGCATCT CGTGAATCAG GTTTTGGACA
  361 CAAGACGAAA GTCGTACGTT CAGTTCGAAG TATCACTTGT
  401 TCGTGATATT CGAGACCGTG AATTCAAGAT TTTCTCAGAC
  441 GCTGGTCGTG TCATGAGACC CGTCTTTACA GTCCATCAGG
  481 AGGATGACTA TGAGAACAAC ATCACCAAGG GACAACTAGT
  521 GTTGACAAAG GAACATGTCA ATAGGCTAGC CCAAGAGCAG
  561 GCAGAGCCAC CTGCCAACCC CGCGGACAAG TTTGGATGGG
  601 ATGGCTTGAT TCGCGAAGGA GCTGTCGAGT ATCTCGACGC
  641 TGAGGAAGAA GAGACAGCCA TGATTTGCAT GACGCCAGAG
  681 GATCTCGAAC TTTACCGTGA GCAAAAGAAT GATGAAGCTA
  721 CACTCACGGA AGAAGAGAAA CGGGCCAAGG CAGAGGCAGA
  761 GAAGAGGGAA CAAGAGGAGG ACCGCAACAA GCGATTGAAG
  801 ACAAAGGTCA ACCCCACAAC TCACATGTAC ACACATTGTG
  841 AGATTCACCC CAGTATGATT CTCGGTATCT GTGCCAGTAT
  881 CATTCCTTTC CCCGATCACA ACCAGGTATG TAGTCCCTTT
  921 GATCACAACA ACCTCAACNN NNNNNNNNNN NNNNNNNNNN
  961 NNNNNNNNNN NNNNNNNNNN NNNNNNNNNN NCAATATTCT
1001 CTACTACCCT CAAAAGCCTC TCGCCACTAC CCGATCCATG
1041 GAGTTCCTCA AGTTCCGTGA ATTGCCAGCT GGTCAAAATG
1081 CCATTGTCGC AATTGCTTGC TACTCAGGTT ATAACCAGGA
1121 AGATTCCGTC ATTATGAACC AGAGTAGTAT TGATCGAGGT
1161 CTGTTCCGAA GTCTGTTCTT CCGATCGTAC TCAGACCAGG
1201 AGAAGAAGGT CGGCCTCAAC TACACTGAGA TCTTTGAGAA
1241 GCCTTTCCAG CAGACAACAC TCCGAATGAA GCATGGAACA
1281 TACGACAAGC TTGACGAGGA TGGTATCGTG GCTCCTGGTG
1321 TCCGTGTGTC TGGTGAAGAT ATCATTATCG GCAAGACTGC
1361 ACCCATCGAC CAAGAAAACC AGGACCTTGG CACAAGGACT
1401 CAATCGCACC AGCGTCGTGA TATCTCGACA CCACTGCGAA
1441 GTACCGAGAA CGGTATCGTT GATCAGGTCA TTCTGACAGT
1481 CAACGCCGAT AACGTCAAGT ACGTCAAGGT TCGAGTACGA
1521 ACAACCAAGA TTCCTCAAAT CGGTGACAAG TTTGCTTCTC
1561 GTCACGGTCA AAAGGGTACA ATCGGTGTTA CATATCGACA
1601 GGAGGATATG CCTTTCAGCC GAGAAGGTCT CACCCCCGAT
1641 ATCATTATCA ACCCTCACGC CATTCCATCG CGAATGACAA
1681 TTGCCCATTT GATTGAGTGT CTCCTTAGCA AGGTTTCAAC
1721 GCTGGAAGGT ATGGAGGGTG ACGCCACACC GTTCACTGAT
1761 GTCACAGTCG ATTCAGTCTC AGAACTTCTG AGGAAGCACG
1801 GTTACCAATC TCGAGGTTTC GAGGTCATGT ACAATGGTCA
1841 CACTGGACGA AAGCTCCGTG CCCAGGTGTT CTTCGGACCT
1881 ACCTACTAC
```

The Fusarium endophyte #40 isolate had the following translation elongation factor 1 alpha (TEF1a) gene sequence (SEQ ID NO:5), which had 100% sequence identity (and 100% coverage) to an EF1-α sequence of two Fusarium commune isolates (NRRL 28058 and NRRL 13816).

```
    1 GACTCACCTT AACGTCGTCG TCATCGGCCA CGTCGACTCT
  41 GGCAAGTCGA CCACTGTGAG TACTCCCCTT GGACGATGAG
  81 CTTATCTGCC ATCGTTAATC CCGACCAAGA CCTGGCGGGG
121 TATTTCTCAA AGGCAATATG CTGATATCGT TTCACAGACC
161 GGTCACTTGA TCTACCAGTG CGGTGGTATC GACAAGCGAA
201 CCATCGAGAA GTTCGAGAAG GTTAGTCACT TTCCCTTCGA
241 TCGCGCGTCC TCTGCCCATC GATTTCCCCT ACGACTCGAA
281 ACCTGCCCGC TACCCCGCTC GAGACCAAAA ATTTTGCGAT
321 ATGACCGTAA TTTTTTTTGG TGGGGCATTT ACCCCGCCAC
361 TCGAGCGACG GGCGCGTTTG CCCTCCTCCC ATTTCCACAA
401 CCTCAATGAG CGCATCGTCA CGTGTCACGC AGTCACTAAC
441 CATTCAATAA TAGGAAGCCG CTGAGCTCGG TAAGGGTTCC
481 TTCAAGTACG CCTGGGTTCT TGACAAGCTC AAGGCCGAGC
521 GTGAGCGTGG TATCACCATC GATATTGCTC TCTGGAAGTT
561 CGAGACTCCT CGCTACTATG TCACCGTCAT TGGTATGTTG
601 TCGCTCATGC TTCATTCTAC TTCTCTTCGT ACTGACATAT
641 CACTCAGACG CTCCCGGTCA CCGTGATTTC ATCAAGAA
```

Based on RPB1 and RPB2 sequences Fusarium endophyte #70 grouped with Fusarium oxysporum NRRL 34936 in a well-supported (posterior probability=1.0) monophyletic group (FIG. 1B). In terms of sequence identity, Fusarium #70 RBP2 sequence (1887 bp) had a 99.75% match with 97.35% overlap to Fusarium oxysporum NRRL 1943. The RPB1 sequence (1470 bp) had a 99.66% match with a 100% coverage to Fusarium oxysporum NRRL 20433. Additionally, the EF1-α sequence (684 bp) had a 99.27% match and 99.56% overlap with Fusarium oxysporum NRRL 1943.

The Fusarium endophyte #70 isolate had the following RPB1 gene sequence (SEQ ID NO:6), which had 99.66% sequence identity (and 100% coverage) to a RPB 1 sequence from Fusarium oxysporum NRRL 20433.

```
      1 TTTTCCTCAC AAAGGAGCAA ATCATGAACT GTATGCTCTG
    41 GGTGCCCAAC TGGGACGGTG TCATTCCTCA ACCCGCTATC
    81 TATAAGCCTC GTCCTCGGTG GACTGGTAAG CAGCTTATCA
  121 GCATGGTTAT CCCTAAGGAG GTTAGCCTGT TCAACGGTAC
  161 GGATTCTGGT GAAAACGCCC CTCTTAAGGA CGAGGGTCTT
  201 CTGATCCAAG CCGGCCAACT GATGTATGGT CTTTTAACTA
  241 AGAAGAACAT TGGTGCTGCT GCGGGTGGTA TTGTGCATAT
  281 CAGCTACAAC GAACTTGGCC CCGAAGGTGC GATGGCTTTC
  321 TTAAACGGTG TCCAGCAGGT TGTCACCTAC TGGCTTCTCA
  361 ACAATGGTCA TAGCATTGGT ATTGGTGATA CAATTCCCGA
  401 TGCGGCGACC ATTGCTAAAG TTCAGGTACA TATTGATGAG
  441 GAAAAGGCTG AAGTTGCCCG CTTGACAGCA ATGGCCACAG
  481 CGAATGAGCT TGAGGCCCTA CCTGGTATGA ACGTTCGTGC
  521 AACCTTCGAA AACAAAGTCT CCATGGCTCT GAACCAGGCC
  561 CGTGATAAGG CTGGTACCAC AACACAGAAG AGTTTGAAGG
  601 ATTCAAACAA CGCTGTCACC ATGGCTTCCT CAGGTTCCAA
  641 GGGTTCATCT ATCAATATTT CTCAAATGAC TGCGCTTGTC
  681 GGTCAGCAAA TTGTCGAAGG CAAGCGTATT CCTTTTGGTT
  721 TCAAGTATCG CACATTACCT CACTTCACCA AGGACGATTA
  761 CTCACCTGAG GCCCGTGGCT TCGTCGAGAA CTCTTACCTC
  801 CGTGGTCTCA CTCCTAGCGA ATTTTTCTTC CACGCCATGG
  841 CTGGTCGAGA AGGTCTCATT GATACTGCAG TCAAGACTGC
  881 CGAAACAGGT TATATCCAGC GACGATTGGT TAAGGCTCTG
  921 GAAGATCTTT CTGCCCGTTA CGATGGAACT GTCCGAAACT
  961 CTCTGGGAGA CATTGTTCAG TTCCTCTATG GTGAAGACGG
1001 TCTTGATGCC ATGATTATTG AGAAACAGAA GTTGGGTATC
1041 CTCAATATGT CAAACTCGGC ATTTGAAAAG AAGTATCGTC
1081 TGGATCTTGC CAACCCCCCG GACTGGTTTA AGCACGACTA
1121 CGAATTCGGT AACGAATTGA CTGGTGACAA GGAATCTATG
1161 GAGTATCTCG ATCAAGAATG GGAAAAGTTG TTGGCTGATC
1201 GCAGACAAGT CCGACAGATC AACAAGGCCA AGGGTAACGA
1241 GGAAATGATG CAACTGCCCC TCAACATCAC TCGCATCATC
1281 GAGTCTGCTA AGCGAGTCTT TAATGTCAAG GCTAATGACC
1321 GAAGCAACTT GCGACCGTCG GAAGTTATTC CAGCTGTGCA
1361 AAACTTGTTG GATAGCATGA AGATTGTTCG TGGTACTGAT
1401 GAAATCTCGA TTGAAGCTGA CGCAAATGCA TCATTTCTCT
1441 TCAAGGCCTT GCTTCGCTCT CGCCTGGCCT
```

The Fusarium endophyte #70 isolate had the following RPB2 gene sequence (SEQ ID NO:7), which had 99.75% sequence identity to a RPB2 from to Fusarium oxysporum NRRL 1943.

```
      1 TGCTTCTACT CTTTCACATT TGCGTCGAAC CAATACTCCC
    41 ATCGGACGAG ATGGTAAATT GGCCAAGCCT CGACAGCTTC
    81 ACAACACTCA CTGGGGTTTG GTGTGTCCTG CCGAAACACC
  121 TGAGGGTCAA GCTTGTGGTC TGGTCAAAAA CTTGTCTCTA
  161 ATGTGTTACG TCAGTGTCGG CTCTCCAGCC GATCCTCTGA
  201 TTGAATTCAT GATCAACAGA GGCATGGAAG TCGTTGAGGA
  241 GTACGAGCCG ACAAGATACC CCCACGCTAC AAAGATTTTC
  281 GTCAACGGTA GCTGGGTTGG TGTTCATGCC GACCCCAAGC
  321 ATCTCGTGAA TCAGGTCTTG GACACAAGAC GAAAGTCTTA
  361 CGTGCAGTTC GAAGTATCAC TTGTTCGTGA TATCCGAGAC
  401 CGTGAATTCA AGATTTTTTC AGACGCTGGC CGTGTCATGA
  441 GACCCGTCTT TACAGTTCAT CAGGAGGATG ACTATGAGAA
  481 CAACATCACC AAGGGACAAC TAGTGTTGAC AAAGGACCAT
  521 GTCAATAGGC TAGCCCAAGA ACAGGCAGAG CCTCCTGCCA
  561 ACCCAGCGGA CAAGTTTGGA TGGGATGGCT TGATCCGCGA
  601 AGGAGCTGTC GAGTATCTCG ATGCTGAGGA AGAAGAGACA
  641 GCCATGATTT GCATGACGCC AGAGGATCTC GAACTTTACC
  681 GTGAGCAAAA GAATGATGAA GCTACACTCA CAGAAGAAGA
  721 GAAACGGGCC AAGCAAGAGG CAGAGAAGAG AGAACAAGAG
  761 GAGGACCGCA ACAAGCGATT GAAGACAAAG GTGAACCCCA
  801 CAACTCACAT GTACACACAT TGTGAGATTC ACCCCAGTAT
  841 GATTCTCGGT ATCTGTGCCA GTATCATTCC TTTCCCCGAT
  881 CACAACCAGG TATGTNNNNN NNNNNNNNNN NNNNNNNNNN
  921 NNNNNNNNNN NNNNNNNNNN NNNNNNNNNN NNNNNNNNNN
  961 NNNNNNNNNN NNNNNNNNNN NNNNNNNNNN NNNNNNNNNN
1001 NNNNNNNNNN NNNNNNNNNN NNNNNNNNNN NNNNNNNNNN
1041 NNNNNNNNNN NNNNNNNNNN NNNNNNNNNN NNNNNNNNNN
1081 NNNNNNNNNN NNNNNNNNNN NNNNNNNNNN NNNNNNNNNN
1121 NNNNNNAGTA GTATTGATCG AGGTTTGTTC CGAAGTCTGT
1161 TCTTCCGATC GTACTCAGAT CAGGAGAAGA AGGTTGGTCT
1121 CAACTACACT GAGATCTTTG AGAAACCTTT CCAGCAGACA
1201 ACGCTTCGAA TGAAGCATGG AACATACGAC AAGCTTGATG
1241 AAGATGGTAT CGTGGCTCCT GGTGTCCGTG TGTCAGGTGA
1281 AGATATCATT ATCGGCAAGA CTGCACCCAT CGACCAAGAA
1321 AACCAGGACC TTGGCACAAG AACTCAATCG CACCAACGTC
1361 GTGATATCTC GACACCACTG CGAAGTACTG AGAACGGTAT
1401 CGTTGATCAA GTCATTCTGA CAGTCAACGC CGATAACGTC
1441 AAGTACGTCA AGGTCCGAGT ACGAACAACC AAGATTCCTC
1481 AAATTGGTGA CAAGTTTGCT TCTCGTCACG GTCAAAAGGG
1521 TACAATCGGT GTTACATATC GACAGGAGGA TATGCCTTTC
1561 AGCCGAGAAG GTCTTACTCC CGATATCATT ATCAACCCTC
1601 ACGCCATTCC ATCGCGAATG ACAATTGCCC ATTTGATTGA
1641 GTGTCTTCTT AGCAAGGTTT CAACGCTGGA AGGTATGGAG
1681 GGTGACGCCA CACCGTTCAC TGATGTCACA GTCGATTCAG
1721 TCTCAGAACT TCTGAGGAAG CACGGTTACC AATCTCGAGG
1761 TTTCGAGGTC ATGTACAACG GTCACACTGG ACGAAAGCTC
1801 CGTGCCCAGG
```

The Fusarium endophyte #70 isolate had the following translation elongation factor 1 alpha (TEF1a) gene sequence (SEQ ID NO:8), which had 99.27% sequence identity and 99.56% overlap with Fusarium oxysporum NRRL 1943.

```
    1 AGACAAGACT CACCTTAACG TCGTCGTCAT CGGCCACGTC
  41 GACTCTGGCA AGTCGACCAC TGTGAGTACT CTCCTTGACA
  81 ATGAGCTTAT CTGCCATCGT CAATCCCGAC CAAGACCTGG
121 CGGGGTATTT CTCAAAGTCA ACATACTGAC ATCGTTTCAC
161 AGACCGGTCA CTTGATCTAC CAGTGCGGTG GTATCGACAA
201 GCGAACCATC GAGAAGTTCG AGAAGGTTAG TTACTTTCCC
241 TTCGATCGCG CGTCTTTTGC CCATCGATTT CCCCTACGAC
281 TCGAAACGTG CCCGCTACCC CTCTCGAGAC CAAAAATTTT
321 GCAATATGAC CGTAATTTTT TTGGTGGGGC ATTTACCCCG
361 CCCCTCGGGT GCCGGGCGCG TTTGCCCTCT TACCATTCTC
401 ACAACCTCAA TGAGCGCATC GTCACGTGTC AAGCAGTCAC
441 TAACCATTCA ACAATAGGAA GCCGCTGAGC TCGGTAAGGG
481 TTCCTTCAAG TACGCCTGGG TTCTTGACAA GCTCAAGGCC
521 GAGCGTGAGC GTGGTATCAC CATCGATATT GCTCTCTGGA
561 AGTTCGAGAC TCCTCGCTAC TATGTCACCG TCATTGGTAT
601 GTTGTCGCTC ATGCTTCATT CTACTTCTCT TCGTACTAAC
641 ACATCACTCA GACGCTCCCG GTCACCGTGA TTTCATCAAG
661 AACATGA
```

Hence, the endophyte isolates used in the methods and compositions described herein are of species Fusarium commune (#40 isolate), Fusarium oxysporum (#70 isolate), and Alternaria destruens (#37 isolate).

### EXAMPLE 3

### Endophytes Inhibit F. graminearum Spread

This Example illustrates that the endophytes described in Example 2 restrict the spread of F. graminearum isolates.

In vitro competitions between the three wheat endophytes and six F. graminearum isolates were evaluated in a first assay that allowed physical contact and in a second assay that did not allow physical contact. The first assay with physical contact was designed to test competition via pre-emption of unoccupied space, whereas the second assay without physical contact tested reduction in the growth of F. graminearum via volatile production.

As shown in FIG. 2A and Table 3, the percentage of surface area in the culture occupied by F. graminearum was significantly lower (P<0.001) in the presence of each of the endophytes than without them.

TABLE 3
Analysis of variance for in vitro competition of three endophytes
against seven Fusarium graminearum isolates

| Factor | Chisq | Df | P-value |
|---|---|---|---|
| Fg isolate | 83.204 | 6 | < 0.001 |
| Endophyte isolate | 1249.141 | 3 | < 0.001 |
| DPIª | 447.127 | 3 | < 0.001 |
| Fg isolate × Endophyte isolate | 107.19 | 18 | < 0.001 |
| Fg isolate × DPI | 14.612 | 18 | 0.6884 |
| Endophyte isolate × DPI | 382.254 | 9 | < 0.001 |
| Fg isolate × Endophyte isolate × DPI | 32.734 | 54 | 0.9902 |
| ^{a}DPI = Days post-inoculation | | | |

The two Fusarium endophytes, F. commune #40 and F. oxysporum #70, restricted the area occupied by F. graminearum isolates by 36.5 to 42.6% after 14 days post-inoculation (dpi) (FIG. 2A). The mean area occupied on the Petri dish of the six F. graminearum isolates tested in the presence of F. commune #40 or F. oxysporum #70 at 14 days post incubation was 12.7±1.06 mm² and 12.1±1.11 mm², respectively, compared to 41.1±3.13 mm² for F. graminearum without endophytes present. A. destruens #37 significantly restricted the growth of F. graminearum, albeit to a lesser extent (18.2±1.42 mm²), but it also preemptively occupied space on the petri dish (FIG. 2A). However, growth restriction was not observed when F. graminearum and endophytes were physically separated indicating that potential volatiles produced by the endophytes did not contribute to competition at a distance (data not shown).

F. commune #40 and A. destruens #37 inhibited the growth of the initial colony of F. graminearum before the endophytes reached the colony edges (FIG. 2B). However, in the presence of F. oxysporum #70, growth of the F. graminearum colony was only slightly less than of the F. graminearum PH-1 isolate without an endophyte (FIG. 2B). Continued growth restriction by the F. commune #40 and A. destruens #37 on all sides defined a smooth-edged PH-1 colony (FIG. 2B). Additionally, morphological observations on the interaction between F. commune #40 and F. graminearum indicated that upon physical contact, a proliferation of F. commune #40 hyphae was observed at the juncture along with an increase in the red pigment observed at 18 dpi compared to F. commune #40 grown without F. graminearum (FIG. 2B). Seven isolates were tested, and all seven F. graminearum isolates were restricted in growth similarly.

### EXAMPLE 4

### Wheat Pre-Treatment with Endophytes

### Decreased Mycotoxins and Increased Seed Weight

This Example illustrates that several species of endophytes were useful for reducing mycotoxins and increasing seed weights in wheat. The endophytes were able to pre-emptively occupy non-colonized portions of the medium and restrict further growth of all six F. graminearum isolates that were tested. These endophytes were then evaluated to see whether they could similarly restrict the growth of F. graminearum PH-1 and reduce disease severity and mycotoxin contamination on wheat heads treated during anthesis.

As shown in Table 4, seed mass following infection from F. graminearum PH-1 was significantly greater when F. commune #40 (P <0.001) or A. destruens #37 (P=0.0023) endophytes were applied to wheat heads before inoculation of the F. graminearum PH-1 pathogen. Pre-colonization with F. commune #40 resulted in near doubling of seed weight when compared to F. graminearum PH-1 inoculated heads without pre-inoculation of endophytes. However, the mean seed weight of F. graminearum PH-1 infected heads pre-inoculated with any of the endophytes was still lower than control heads without inoculation with F. graminearum PH-1 (Table 4). All three endophytes significantly reduced DON and 15A-DON levels in the seeds compared to the F. graminearum PH-1 infected heads (Table 4).

TABLE 4
Seed Mass and DON Accumulation in Wheat Treated with
Endophytes and Infected with F. graminearum (Fg)

| | Mean seed mass | | DON | | 15-ADON | |
|---|---|---|---|---|---|---|
| Treatment | per seed (mg) ± SE^{a} | | (mg/kg)^{b} | | (mg/kg) | |
| Control | 29.2 ± 2.40 | a | 0 | a | 0 | a |
| Fg | 11.3 ± 0.90 | d | 87.6 ± 18.8 | b | 2.7 ± 0.437 | b |
| Fg + A. destruens #37 | 18.1 ± 1.44 | bc | 25.3 ± 5.30 | c | 0.8 ± 0.170 | c |
| Fg + F. commune #40 | 21.8 ± 1.69 | b | 17.6 ± 4.30 | c | 0.7 ± 0.174 | c |
| Fg + F. oxysporum #70 | 16.1 ± 1.10 | cd | 19.2 ± 4.58 | c | 0.7 ± 0.165 | c |

^{a}Mean values followed by the same letters within columns are not significantly different according to Tukey's honest significant difference (α ≤ 0.05).
^{b}Mean values followed by the same letters within columns are not significantly different according to pairwise Wilcoxon Ranked sum test with Bonferroni correction (α = 0.05)

### EXAMPLE 5

### Wheat Pre-Treatment With Endophytes Increased Plant Mass

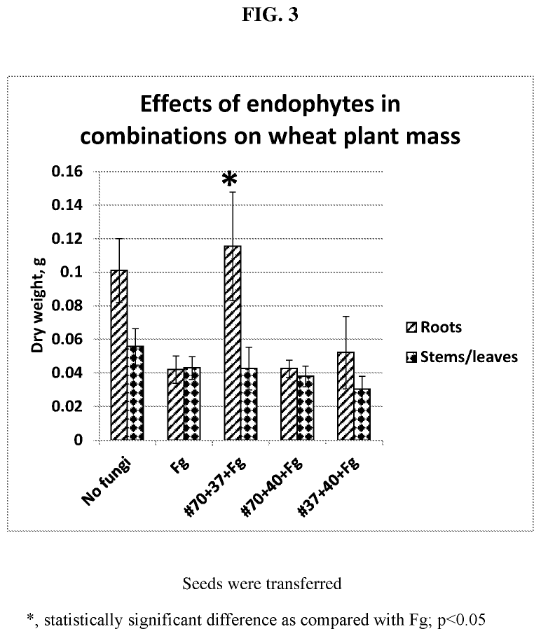

This Example illustrates that several species of endophytes were useful for increasing the weight of wheat plants. As shown in FIG. 3, wheat plants that were grown with F. graminearum (Fg) had a lower weight of roots, stems, and leaves compared to wheat plants grown without F. graminearum (“No fungi”). Wheat plants grown with F. graminearum and the beneficial fungus Alternaria destruens (#37) and Fusarium oxysporum (#70) had roots that had a dry weight that is on average three times higher than wheat plants grown with F. graminearum alone. Wheat plants grown with F. graminearum, F. commune (#40), and either Alternaria destruens (#37) or Fusarium oxysporum (#70) had roots with a dry weight similar to wheat plants grown with F. graminearum alone. These results indicate that the combination of Alternaria destruens (#37) and Fusarium oxysporum (#70) may have a synergistic effect on inhibiting F. graminearum and boosting wheat root mass.

### REFERENCES

Adhikari, B. N., Bandyopadhyay, R., and Cotty, P. J. 2016. Degeneration of aflatoxin gene clusters in Aspergillus flavus from Africa and North America. AMB Express. 6:62.

Alexander, N. J., McCormick, S. P., Waalwijk, C., van der Lee, T., and Proctor, R. H. 2011. The genetic basis for 3-ADON and 15-ADON trichothecene chemotypes in Fusarium. Fungal Genet. Biol. 48:485-495.

Anderson, N. R., Freije, A. N., Bergstrom, G. C., Bradley, C. A., Cowger, C., Faske, T., et al. 2020. Sensitivity of Fusarium graminearum to Metconazole and Tebuconazole Fungicides Before and After Widespread Use in Wheat in the United States. Plant Health Prog. 21:85-90.

Atehnkeng, J., Donner, M., Ojiambo, P. S., Ikotun, B., Augusto, J., Cotty, P. J., et al. 2016. Environmental distribution and genetic diversity of vegetative compatibility groups determine biocontrol strategies to mitigate aflatoxin contamination of maize by Aspergillus flavus. Microb. Biotechnol. 9:75-88.

Bandyopadhyay, R., Ortega-Beltran, A., Akande, A., Mutegi, C., Atehnkeng, J., Kaptoge, L., et al. 2016. Biological control of aflatoxins in Africa: current status and potential challenges in the face of climate change. World Mycotoxin J. 9:771-789.

Bates, D., Mächler, M., Bolker, B., and Walker, S. 2015. Fitting Linear Mixed-Effects Models Using lme4. Journal of Statistical Software, Articles. 67:1-48.

Broders, K. D., Lipps, P. E., Paul, P. A., and Dorrance, A. E. 2007. Evaluation of Fusarium graminearum Associated with Corn and Soybean Seed and Seedling Disease in Ohio. Plant Dis. 91:1155-1160.

Brown, D. W., Proctor, R. H., Dyer, R. B., and Plattner, R. D. 2003. Characterization of a fusarium 2-gene cluster involved in trichothecene C-8 modification. J. Agric. Food Chem. 51:7936-7944.

Constantin, M. E., de Lamo, F. J., Vlieger, B. V., Rep, M., and Takken, F. L. W. 2019. Endophyte-Mediated Resistance in Tomato to Fusarium oxysporum is independent of ET, JA, and SA. Front. Plant Sci. 10:979.

Cotty, P. J. 1994. Influence of field application of an atoxigenic strain of Aspergillus flavus on the populations of A. flavus infecting cotton bolls and on the aflatoxin content of cottonseed. Phytopathology. 84:1270.

Cowger, C., and Arellano, C. 2013. Fusarium graminearum infection and deoxynivalenol concentrations during development of wheat spikes. Phytopathology. 103:460-471.

Cuomo, C. A., Güldener, U., Xu, J.-R., Trail, F., Turgeon, B. G., Di Pietro, A., et al. 2007. The Fusarium graminearum genome reveals a link between localized polymorphism and pathogen specialization. Science. 317:1400-1402.

Dvorska, J. E., Surai, P. F., Speake, B. K., and Sparks, N. H. 2001. Effect of the mycotoxin aurofusarin on the antioxidant composition and fatty acid profile of quail eggs. Br. Poult. Sci. 42:643-649.

Frandsen, R. J. N., Schütt, C., Lund, B. W., Staerk, D., Nielsen, J., Olsson, S., et al. 2011. Two novel classes of enzymes are required for the biosynthesis of aurofusarin in Fusarium graminearum. J. Biol. Chem. 286:10419-10428.

Gaffoor, I., Brown, D. W., Plattner, R., Proctor, R. H., Qi, W., and Trail, F. 2005. Functional analysis of the polyketide synthase genes in the filamentous fungus Gibberella zeae (anamorph Fusarium graminearum). Eukaryot. Cell. 4:1926-1933.

Gaffoor, I., and Trail, F. 2006. Characterization of two polyketide synthase genes involved in zearalenone biosynthesis in Gibberella zeae. Appl. Environ. Microbiol. 72:1793-1799.

Gdanetz, K., and Trail, F. 2017. The Wheat Microbiome Under Four Management Strategies, and Potential for Endophytes in Disease Protection. Phytobiomes Journal. 1:158-168.

Harris, L. J., Desjardins, A. E., Plattner, R. D., Nicholson, P., Butler, G., Young, J. C., et al. 1999. Possible Role of Trichothecene Mycotoxins in Virulence of Fusarium graminearum on Maize. Plant Dis. 83:954-960.

Hofstetter, V., Miadlikowska, J., Kauff, F., and Lutzoni, F. 2007. Phylogenetic comparison of protein-coding versus ribosomal RNA-coding sequence data: a case study of the Lecanoromycetes (Ascomycota). Mol. Phylogenet. Evol. 44:412-426.

Ishii, H., and Hollomon, D. W. 2015. Fungicide Resistance in Plant Pathogens: Principles and a Guide to Practical Management. Springer.

Kim, J.-E., Jin, J., Kim, H., Kim, J.-C., Yun, S.-H., and Lee, Y.-W. 2006. GIP2, a putative transcription factor that regulates the aurofusarin biosynthetic gene cluster in Gibberella zeae. Appl. Environ. Microbiol. 72:1645-1652.

Kim, Y.-T., Lee, Y.-R., Jin, J., Han, K.-H., Kim, H., Kim, J.-C., et al. 2005. Two different polyketide synthase genes are required for synthesis of zearalenone in Gibberella zeae. Mol. Microbiol. 58:1102-1113.

de Lamo, F. J., and Takken, F. L. W. 2020. Biocontrol by Using Endophyte-Mediated Resistance. Front. Plant Sci. 11:37.

Lawrence, D. P., Gannibal, P. B., Peever, T. L., and Pryor, B. M. 2013. The sections of Alternaria: formalizing species-group concepts. Mycologia. 105:530-546.

Leonard, K. J., and Bushnell, W. R. 2003. Fusarium Head Blight of Wheat and Barley. Amer Phytopathological Society.

Liu, Y. J., Whelen, S., and Hall, B. D. 1999. Phylogenetic relationships among ascomycetes: evidence from an RNA polymerse II subunit. Mol. Biol. Evol. 16:1799-1808.

Llorens, E., Sharon, O., Camañes, G., García-Agustin, P., and Sharon, A. 2019. Endophytes from wild cereals protect wheat plants from drought by alteration of physiological responses of the plants to water stress. Environ. Microbiol. Available at: http://dx.doi.org/10.1111/1462-2920.14530.

Matarese, F., Sarrocco, S., Gruber, S., Seidl-Seiboth, V., and Vannacci, G. 2012. Biocontrol of Fusarium head blight: interactions between Trichoderma and mycotoxigenic Fusarium. Microbiology. 158:98-106.

McMullen, M., Bergstrom, G., De Wolf, E., Dill-Macky, R., Hershman, D., Shaner, G., et al. 2012. A Unified Effort to Fight an Enemy of Wheat and Barley: Fusarium Head Blight. Plant Dis. 96:1712-1728.

Medentsev, A. G., Kotik, A. N., Trufanova, V. A., and Akimenko, V. K. 1993. [Identification of aurofusarin in Fusarium graminearum isolates, causing a syndrome of worsening of egg quality in chickens]. Prikl. Biokhim. Mikrobiol. 29:542-546.

O'Donnell, K., Kistler, H. C., Cigelnik, E., and Ploetz, R. C. 1998. Multiple evolutionary origins of the fungus causing Panama disease of banana: concordant evidence from nuclear and mitochondrial gene genealogies. Proc. Natl. Acad. Sci. U. S. A. 95:2044-2049.

O'Donnell, K., Rooney, A. P., Proctor, R. H., Brown, D. W., McCormick, S. P., Ward, T. J., et al. 2013. Phylogenetic analyses of RPB1 and RPB2 support a middle Cretaceous origin for a clade comprising all agriculturally and medically important fusaria. Fungal Genet. Biol. 52:20-31.

O'Donnell, K., Sutton, D. A., Rinaldi, M. G., Sarver, B. A. J., Balajee, S. A., Schroers, H.-J., et al. 2010. Internet-accessible DNA sequence database for identifying fusaria from human and animal infections. J. Clin. Microbiol. 48:3708-3718.

Paul, P. A., Salgado, J. D., Bergstrom, G., Bradley, C. A., Byamukama, E., Byrne, A. M., et al. 2019. Integrated Effects of Genetic Resistance and Prothioconazole+Tebuconazole Application Timing on Fusarium Head Blight in Wheat. Plant Dis. 103:223-237.

Pestka, J. J. 2010. Deoxynivalenol: mechanisms of action, human exposure, and toxicological relevance. Arch. Toxicol. 84:663-679.

Probst, C., Bandyopadhyay, R., Price, L. E., and Cotty, P. J. 2011. Identification of Atoxigenic Aspergillus flavus Isolates to Reduce Aflatoxin Contamination of Maize in Kenya. Plant Dis. 95:212-218.

Proctor, R. H., Hohn, T. M., and McCormick, S. P. 1995. Reduced virulence of Gibberella zeae caused by disruption of a trichothecene toxin biosynthetic gene. Mol. Plant. Microbe. Interact. 8:593-601.

Reeb, V., Lutzoni, F., and Roux, C. 2004. Contribution of RPB2 to multi-locus phylogenetic studies of the euascomycetes (Pezizomycotina, Fungi) with special emphasis on the lichen-forming Acarosporaceae and evolution of polyspory. Mol. Phylogenet. Evol. 32:1036-1060.

Ripa, F. A., Cao, W.-D., Tong, S., and Sun, J.-G. 2019. Assessment of Plant Growth Promoting and Abiotic Stress Tolerance Properties of Wheat Endophytic Fungi. Biomed Res. Int. 2019:6105865.

Rodriguez, R. J., White, J. F., Jr, Arnold, A. E., and Redman, R. S. 2009. Fungal endophytes: diversity and functional roles. New Phytol. 182:314-330.

Ronquist, F., Teslenko, M., van der Mark, P., Ayres, D. L., Darling, A., Höhna, S., et al. 2012. MrBayes 3.2: efficient Bayesian phylogenetic inference and model choice across a large model space. Syst. Biol. 61:539-542.

Saito, H., Sasaki, M., Nonaka, Y., Tanaka, J., Tokunaga, T., Kato, A., et al. 2021. Spray Application of Nonpathogenic Fusaria onto Rice Flowers Controls Bakanae Disease (Caused by Fusarium fujikuroi) in the Next Plant Generation. Appl. Environ. Microbiol. 87 Available at: aem.asm.org/content/87/2/e01959-20 [Accessed Mar. 30, 2021].

Schindelin, J., Arganda-Carreras, I., Frise, E., Kaynig, V., Longair, M., Pietzsch, T., et al. 2012. Fiji: an open-source platform for biological-image analysis. Nat. Methods. 9:676-682.

Schöneberg, A., Musa, T., Voegele, R. T., and Vogelgsang, S. 2015. The potential of antagonistic fungi for control of Fusarium graminearum and Fusarium crookwellense varies depending on the experimental approach. J. Appl. Microbiol. 118:1165-1179.

Sondergaard, T. E., Fredborg, M., Oppenhagen Christensen, A.-M., Damsgaard, S. K., Kramer, N. F., Giese, H., et al. 2016. Fast Screening of Antibacterial Compounds from Fusaria. Toxins. 8 Available at: dx.doi.org/10.3390/toxins8120355.

Spolti, P., de Jorge, B. C., and Del Ponte, E. M. 2012. Sensitivity of Fusarium graminearum causing head blight of wheat in Brazil to tebuconazole and metconazole fungicides. Tropical Plant Pathology. 37:419-423 Available at: dx.doi.org/10.1590/s1982-56762012000600007.

Sridhar, K. R. 2019. Diversity, Ecology, and Significance of Fungal Endophytes. In Endophytes and Secondary Metabolites, ed. Sumita Jha. Cham: Springer International Publishing, p. 61-100.

Trail, F. 2009. For blighted waves of grain: Fusarium graminearum in the post-genomics era. Plant Physiol. 149:103-110.

Trail, F., and Common, R. 2000. Perithecial Development by Gibberella zeae: A Light Microscopy Study. Mycologia. 92:130 Available at: dx.doi.org/10.2307/3761457. Website. Available at: www3.epa.gov/pesticides/chem_search/reg_actions/registration/fs_PC-028301_05-Apr-05.pdf) [Accessed Mar. 30, 2021].

Woudenberg, J. H. C., Seidl, M. F., Groenewald, J. Z., de Vries, M., Stielow, J. B., Thomma, B., et al. 2015. Alternaria section Alternaria: Species, formae speciales or pathotypes? Stud. Mycol. 82:1-21.

Yin, Y., Liu, X., Li, B., and Ma, Z. 2009. Characterization of sterol demethylation inhibitor-resistant isolates of Fusarium asiaticum and F. graminearum collected from wheat in China. Phytopathology. 99:487-497.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements of the invention are intended to describe and summarize various embodiments of the invention according to the foregoing description in the specification.

### Statements

1. A method comprising administering or applying to seeds, plants, crops, soils, or combinations thereof a composition comprising one or more of the following endophytes: Alternaria destruens, Fusarium commune, Fusarium oxysporum, or a combination thereof.

2. The method of statement 1, wherein the endophytes comprise conidia, spores, propagules, or a combination thereof.

3. The method of statement 1 or 2, wherein the endophytes are administered to plant parts comprising roots, buds, flowers, leaves, fruits, seeds, or a combination thereof.

4. The method of statement 1, 2 or 3, wherein the endophytes are administered to whole plants.

5. The method of any of statements 1-4, wherein the Alternaria destruens comprise nucleic acids or DNA copies thereof with at least 70%, at least 75%, at least 80%, at least 85%, at least 90%, at least 95%, at least 96%, at least 97%, at least 98%, at least 99% sequence identity to any of SEQ ID NO:1 or 2.

6. The method of any of statements 1-5, wherein the Alternaria destruens comprise Alternaria destruens CBS 121454, Alternaria lini CBS 106.34, or a combination thereof.

7. The method of any of statements 1-6, wherein the Fusarium commune comprise nucleic acids or DNA copies thereof with at least 70%, at least 75%, at least 80%, at least 85%, at least 90%, at least 95%, at least 96%, at least 97%, at least 98%, at least 99% sequence identity to SEQ ID NO:3-5.

8. The method of any of statements 1-7, wherein the Fusarium commune comprises Fusarium commune NRRL 28387, Fusarium commune NRRL 13816, Fusarium commune NRRL 28058, or a combination thereof.

9. The method of any of statements 1-8, wherein the Fusarium oxysporum comprise nucleic acids or DNA copies thereof with at least 70%, at least 75%, at least 80%, at least 85%, at least 90%, at least 95%, at least 96%, at least 97%, at least 98%, at least 99% sequence identity to SEQ ID NO:6-8.

10. The method of any of statements 1-9, wherein the Fusarium oxysporum comprises Fusarium oxysporum NRRL 20433, Fusarium oxysporum NRRL 1943, or a combination thereof.

11. The method of any of statements 5-9, wherein the nucleic acids are genomic nucleic acids, transcripts, or mRNAs.

12. The method of any of statements 1-11, wherein composition further comprises a carrier.

13. The method of statement 12, wherein the carrier is an aqueous or oil-based carrier.

14. The method of statement 12, wherein the carrier is a dry dispersant.

15. The method of any of statements 1-14, wherein the seeds, plants, crops, or combinations thereof comprise wheat, barley, rye, oats, millet, triticale, maize, rice, sorghum, or combinations thereof.

16. The method of any of statements 1-15, wherein the soils are prepared for planting wheat, barley, rye, oats, millet, triticale, maize, rice, sorghum, or combinations thereof.

17. The method of any of statements 1-16, wherein the composition is administered or applied in an amount sufficient to inhibit the spread and/or mycotoxin production of Fusarium graminearum.

18. The method of any of statements 1-17, wherein the composition is administered or applied in an amount sufficient to inhibit the spread and/or mycotoxin production of Fusarium graminearum by at least 5%, or at least 10%, or at least 15%, or at least 20%, or at least 25%, or at least 30%, or at least 35%, or at least 40%, or at least 45%, or at least 50%, or at least 55%, or at least 60%, or at least 70%, or at least 75%.

19. The method of any of statements 1-18, wherein the composition is administered or applied in an amount about 10⁹-10¹⁶ CFU per hectare, or about 10¹²-10¹⁴ CFU per hectare.

20. A composition comprising a carrier conidia, spores, propagules, or a combination thereof from at least two of the following endophytes: Alternaria destruens, Fusarium commune, Fusarium oxysporum, or a combination thereof.

21. The composition of statement 20, wherein the conidia, spores, propagules, or a combination thereof are dried and encapsulated.

22. The composition of statement 20 or 21, wherein the carrier comprises corn starch, rice flour, talc, diatomaceous earth, kaolin, plant oil, or a combination thereof.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A method comprising administering or applying to seeds, plants, plant parts, crops, soils, or combinations thereof, a composition comprising one or more of the following endophytes: Alternaria destruens, Fusarium commune, Fusarium oxysporum, or a combination thereof.

2. The method of claim 1, wherein the endophytes comprise conidia, spores, propagules, or a combination thereof.

3. The method of claim 1, wherein the endophytes are administered to whole plants.

4. The method of claim 1, wherein the endophytes are administered to plant parts comprising buds, flowers, fruits, roots, seeds, leaves, or a combination thereof.

5. The method of claim 1, wherein the endophytes are administered or incorporated into soils in preparation for planting.

6. The method of claim 1, wherein the Alternaria destruens comprise nucleic acids or DNA copies thereof with at least 70%, at least 75%, at least 80%, at least 85%, at least 90%, at least 95%, at least 96%, at least 97%, at least 98%, at least 99% sequence identity to any of SEQ ID NO:1 or 2.

7. The method of claim 1, wherein the Alternaria destruens comprise Alternaria destruens CBS 121454, Alternaria lini CBS 106.34, or a combination thereof.

8. The method of claim 1, wherein the Fusarium commune comprise nucleic acids or DNA copies thereof with at least 70%, at least 75%, at least 80%, at least 85%, at least 90%, at least 95%, at least 96%, at least 97%, at least 98%, at least 99% sequence identity to SEQ ID NO:3-5.

9. The method of claim 1, wherein the Fusarium commune comprises Fusarium commune NRRL 28387, Fusarium commune NRRL 13816, Fusarium commune NRRL 28058, or a combination thereof.

10. The method of claim 1, wherein the Fusarium oxysporum comprise nucleic acids or DNA copies thereof with at least 70%, at least 75%, at least 80%, at least 85%, at least 90%, at least 95%, at least 96%, at least 97%, at least 98%, at least 99% sequence identity to SEQ ID NO:6-8.

11. The method of claim 1, wherein the Fusarium oxysporum comprises Fusarium oxysporum NRRL 20433, Fusarium oxysporum NRRL 1943, or a combination thereof.

12. The method of claim 1, which is administered in an amount sufficient to inhibit the spread and/or mycotoxin production of Fusarium graminearum by at least 5%, or at least 10%, or at least 15%, or at least 20%, or at least 25%, or at least 30%, or at least 35%, or at least 40%, or at least 45%, or at least 50%, or at least 55%, or at least 60%, or at least 70%, or at least 75%.

13. The method of claim 1, wherein the seeds, plants, crops, or combinations thereof comprise wheat, barley, rye, oats, millet, triticale, maize, rice, sorghum, or combinations thereof.

14. The method of claim 1, wherein the soils are prepared for planting wheat, barley, rye, oats, millet, triticale, maize, rice, sorghum, or combinations thereof.

15. The method of claim 1, wherein the composition is administered or applied in an amount about 10⁹-10¹⁶ CFU per hectare, or about 10¹²-10¹⁴ CFU per hectare.

16. A composition comprising a carrier conidia, spores, propagules, or a combination thereof from at least two of the following endophytes: Alternaria destruens, Fusarium commune, Fusarium oxysporum, or a combination thereof.

17. The composition of claim 16, wherein the conidia, spores, propagules, or a combination thereof are dried and encapsulated.

18. The composition of claim 16, wherein the carrier comprises corn starch, rice flour, talc, diatomaceous earth, kaolin, plant oil, or a combination thereof.
